(19) United States
(12) Patent Application Publication
(10) Pub. No.: US 2009/0246191 A1
(43) Pub. Date: Oct. 1, 2009

(54) Preparation of Purified Covalently Cross-linked Abeta Oligomers and Uses Thereof

(75) Inventors: Brian O'Nuallain, Dublin (IE); Alan Solomon, Knoxville, TN (US); Jonathan S. Wall, Knoxville, TN (US); Luis Acero, Knoxville, TN (US); Angela Williams, Knoxville, TN (US)

Correspondence Address: MORGAN LEWIS & BOCKIUS LLP, 1111 PENNSYLVANIA AVENUE NW, WASHINGTON, DC, 20004, US

(73) Assignee: University of Tennessee Research Foundation, Knoxville, TN (US)

(21) Appl. No.: 12/251,277

(22) Filed: Oct. 14, 2008

Related U.S. Application Data
(60) Provisional application No. 60/979,282, filed on Oct. 11, 2007.

(51) Int. Cl. A61K 39/395; C12P 21/00; C07K 1/00; C07K 16/18; G01N 33/53

(52) U.S. Cl. 424/130.1; 435/68.1; 530/402; 530/413; 530/387.1; 435/7.1

(57) ABSTRACT

The present invention provides a method of purifying cross-linked oligomers. The purified cross-linked oligomers are useful as immunogen for generating and isolating cross-linked oligomer reactive antibodies. The cross-linked oligomer reactive antibodies are useful for detecting amyloid deposition and for diagnosing and treating diseases and conditions associated with amyloid deposition.

### RELATED APPLICATION

This application claims the benefit of U.S. Provisional Application 60/979,282, filed Oct. 11, 2007, which are herein incorporated by reference in its entirety.

### FIELD OF THE INVENTION

The present invention relates to the development of methods and tools effective for treating, preventing, and diagnosing amyloidosis. Specifically, the present invention is directed to methods of treating, preventing, and diagnosing amyloidosis comprising using antibodies.

### BACKGROUND OF THE INVENTION

Amyloidosis

Amyloidosis is a pathologic process in which normally soluble proteins of diverse chemical composition are deposited as fibrils in the brain, heart, liver, pancreas, kidneys, nerves, and other vital tissues, leading to organ failure and, eventually, death. This disorder represents an ever increasing, devastating medical and socioeconomic problem. Among the illnesses associated with amyloid are Alzheimer's disease (AD), adult-onset (type 2) diabetes, certain forms of cancer (multiple myeloma and the related plasma cell disorder, primary [AL] amyloidosis) and inherited disorders (familial amyloidotic polyneuropathy, etc.), chronic inflammation (rheumatoid arthritis, tuberculosis, etc.), and the transmissible spongiform prion-associated encephalopathies. Additionally, amyloid deposition is an invariable consequence of aging (senile systemic amyloidosis, cataracts, etc.) (Benson et al., 2001; Ross et al., 2004; Enqvist et al., 2003; Meehan et al., 2004).

To date, many different amyloidogenic proteins have been identified (Table 1) (Westermark et al., 2002), but irrespective of their varied amino acid sequences, sources of origin, or biologic functions, all types of fibrils have virtually identical tinctorial and ultrastructural features, i.e., when stained by the diazobenzadine sulfonate dye Congo red and examined by polarizing microscopy, they exhibit a characteristic green birefringence (Westermark et al., 2002) and their interaction with thioflavin T (ThT) results in a 120 nm red shift in the excitation spectrum of this benzothiazole compound (LeVine et al., 1995).

TABLE 1
Amyloid Nomenclature: Amyloid fibril proteins
and their precursors in humans*

| Amyloid | Protein | Syndrome or Involved Tissue |
|---|---|---|
| Protein | Precursor | (Systemic [S] or Localized [L] |
| AL | Immunoglobulin | Primary (S, L), |
| | light chain | Myeloma-associated |
| AH | Immunoglobulin | Primary (S, L), |
| | heavy chain | Myeloma-associated |
| ATTR | Transthyretin | Familial (S), Senile systemic, |
| | | Tenosynovium (L?) |
| Aβ₂M | β₂-microblobulin | Hemodialysis (S), Joints (L?) |
| AA | (Apo)serum AA | Secondary, reactive (S) |
| AapoAI | Apolipoprotein AI | Familial (S), Aortic (L) |
| AApo AII | Apolipoprotein AII | Familial (S) |
| Agel | Gelsolin | Familial (S) |
| Alys | Lyosozyme | Familial (S) |
| Afib | Fibrinogen α-chain | Familial (S) |
| Acys | Cystatin C | Familial (S) |
| Abri | ABriPP | Familial dementia, British (L, S?) |
| Adan | ADanPP | Familial dementia, Danish (L) |
| Aβ | Aβ protein precursor | Alzheimer's disease, aging (L) |
| AprP | Prion protein | Spongiform encephalopathies (L) |
| ACal | (Pro)calcitonin | C-cell thyroid tumors (L) |
| AIAPP | Islet amyloid polypeptide | Islets of Langerhans (L), |
| | | Insulinomas |
| AANF | Atrial natriuetic factor | Cardiac atria (L) |
| APro | Prolactin | Aging pituitary (L), |
| | | Prolactinomas |
| Alns | Insulin | latrogenic (L) |
| Amed | Lactadherin | Senile aortic, media (L) |
| AKer | Kerato-epithelin | Cornea; Familial (L) |
| A(Pin) | Unknown | Pindborg tumors (L) |
| ALac | Lactoferrin | Cornea; Familial (L) |

*Modified from Westermark et al., 2002

When negatively stained with uranyl acetate and viewed by electron microscopy, the fibrils are ˜10 nm in diameter, of indeterminate length, and consist of 2-5, often twisted, filaments arranged in parallel, with surface cross-banding patterns indicative of a helical structure (Goldsbury et al., 1997). Moreover, amyloid fibris have an x-ray fiber diffraction pattern that includes dominant structural repeat reflections at ˜4.7 Å on the meridian and spacings of ˜10 Å on the equator. These characteristics are consistent with a cross β-conformation and indicate that the amyloid polypeptide is organized, with respect to the fibril axis, as perpendicular β strands (Serpell et al., 2000). This cross-β pleated configuration (which has been confirmed by solid-state nuclear magnetic resonance [NMR] (Landsbury et al., 1995), Fourier transfer infrared [FTIR] spectroscopy (Seshadri et al., 1999), and x-ray crystallography (Makin et al., 2005)) accounts for the typical birefringent and morphologic features of amyloid.

Polyclonal and monoclonal antibodies (mAb) have been generated that specifically recognize antigenic determinants expressed on amyloid fibrils or soluble oligomeric assembly intermediates, but not the native precursor proteins (Franklin et al., 1972; Linke et al., 1973; Gaskin et al., 1993; Gevorkian et al., 2004; Hrncic et al., 2000; O'Nuallain et al., 2002, 2004; Goldsteins et al., 1999; Kayed et al., 2003; Paramithiotis et al., 2003; Curin-Serbec et al., 2004; Dumoulin et al., 2004; Glabe et al., 2004). Additionally, IgG or IgM mAbs prepared against light chain (LC) or amyloid β peptide (Aβ) fibrils have been found to react with those formed from unrelated amyloidogenic precursors, including β₂-microglobulin (β₂M), serum amyloid A protein (SAA), islet amyloid polypeptide (IAPP), transthyretin (TTR), and polyglutamine (polyGin) (Hrncic et al., 2000; O'Nuallain et al., 2002). The demonstration that amyloid fibrils, regardless of protein composition, share generic conformational epitopes has provided additional evidence for the presence of structural commonalities among these molecules.

In summary, amyloid is not a uniform deposit and may be composed of unrelated proteins. Various proteins have been identified as capable of forming amyloid in human diseases, for example, immunoglobulin light chains, serum amyloid A protein, β2-microglobulin, transthyretin, cystatin C variant, gelsolin, procalcitonin, PrP protein, amyloid β-protein, ApoA1, and lysozyme. Although these proteins are unrelated, the fibrils which they form have the following common biological properties: 1) they possess a β-pleated sheet secondary structure; 2) they are insoluble aggregates; 3) they exhibit green birefringence after Congo red staining; and 4) they possess a characteristic unbranching fibrillar structure when observed under an electron microscope.

Amyloid Reactive Antibodies

Passive immunotherapy using fibril-reactive mAbs has been shown experimentally to reduce amyloid formation and also accelerate amyloidolysis. WO 2006/113347 discloses that human sera, as well as various sources of pooled human IgG, including pharmacologic formulations of immune globulin intravenous (IGIV), contain antibodies that specifically recognize fibrils formed from light chains (LC) and other amyloidogenic precursor proteins, including serum amyloid A (SAA), transthyretin (TTR), islet amyloid polypeptide (IAPP), and amyloid β 1-40 peptide (Aβ), but notably, do not react with these molecules in their native non-fibrillar forms. WO 2006/113347 shows that after isolation of the antibodies from IGIV via fibril-conjugated affinity column chromatography, the EC50 binding value for LC and Aβ fibrils was ˜15 nM—a magnitude ˜200- and 70-times less than that of the unbound fraction and unfractionated product, respectively. Comparable reactivity was found in the case of those formed from SAA, TTR, and IAPP. The purified antibodies immunostained human amyloid tissue deposits and additionally, could inhibit fibrillogenesis, as shown in fibril formation and extension assays. Most importantly, in vivo reactivity was evidenced in a murine model when the enriched antibodies were used to image amyloid, as well as expedite its removal. WO 2006/113347 shows that fibril affinity-purified IGIV has potential as a diagnostic and therapeutic agent for patients with amyloid-associated disease.

Moreover, there is increasing evidence in AD that Aβ fibril assembly intermediates, including soluble cross-linked Aβ oligomers, are neurotoxic and represent the pathogenic culprits in this disorder (Lee et al., 2006; Hardy et al., 2002; Klyubin et al., 2005; Watson et al., 2005). It has been shown that a conformation-specific monoclonal antibody (mAb) directed against cross-linked Aβ oligomers improved learning and memory in Aβ precursor protein (APP) transgenic mice (Lee et al., 2006). However, the lack of reproducible methods to prepare and purify stable Aβ oligomers has been a limiting factor in using producing such antibodies for potential therapeutic or diagnostic uses for AD.

Accordingly, there is a need to develop a reproducible method for preparing and purifying anti-oligomer antibodies useful in the treatment of amyloidoses, such as AD. As an example, there is a need to obtain antibodies against cross-linked Aβ oligomers.

### SUMMARY OF THE INVENTION

The present invention provides a method of preparing cross-linked oligomers comprising incubating an amyloidogenic peptide or protein with horseradish peroxidase (HRP) to form a solution of cross-linked oligomers; adding copper ions to the solution to precipitate the cross-linked oligomers; and isolating the cross-linked oligomers. The peptide may be solubilized prior to incubation with HRP by sequential exposure to trifluoroacetic acid (TFA) and 1,1,1,3,3,3,-hexafluoro-2-propanol (HFIP) or by dissolving the peptide in sodium hydroxide (NaOH) or other appropriate solvents.

In one embodiment, the HRP is conjugated to a matrix. The HRP conjugated matrix may be treated with a blocking agent prior to incubating with the peptide. The blocking agent may be bovine serum albumin (BSA), gelatin, or other appropriate reagents.

The method of the present invention may further comprise incubating the precipitated cross-linked oligomers under conditions allowing removal of residual HRP and copper ions, prior to isolating the cross-linked oligomers. Guanidine hydrochloride and ethylene diamine tetra-acetic acid (EDTA) or other appropriate reagents may be added to the precipitated cross-linked oligomers to allow removal of residual HRP. The precipitated oligomers may be resolubilized in PBS with added EDTA and centrifuged subsequently to remove residual impurities from the supernatant prior to isolating the soluble cross-linked oligomers.

The present invention also provides a method of preparing soluble cross-linked oligomers comprising solubilizing the amyloidogenic tyrosine containing peptide or protein by sequential exposure to trifluoroacetic acid (TFA) and 1,1,1,3,3,3,-hexafluoro-2-propanol (HFIP) or by dissolving the peptide in sodium hydroxide (NaOH); and incubating the peptide with HRP to form a solution of cross-linked oligomers.

The peptide used to produce the oligomer may be any amyloidogenic peptide or protein. The peptide or protein may comprise one or more tyrosine residues. The peptide may be the Aβ peptide. The oligomer may contain tyrosine cross-linking, for example, dityrosine cross-linking. The cross-linking may be intra-molecular or inter-molecular. For example, the peptide may be the Aβ, and the tyrosine cross-linking may be between two Aβ peptides.

In one embodiment, the present invention provides an affinity purification matrix comprising cross-linked oligomers. The cross-linked oligomers may be conjugated to the matrix. The cross-linked oligomers may be any amyloidogenic oligomer useful as a ligand in affinity purification. In one embodiment, the oligomers may be cross-linked Aβ oligomers, also known as soluble cross-linked β-amyloid protein species (CAPS). The matrix may comprise any appropriate resin used in affinity purification. The affinity matrix may comprise sepharose.

Moreover, the invention provides a method of preparing an affinity purification matrix comprising purifying the cross-linked oligomers as described above; preparing an affinity purification matrix; and conjugating the cross-linked oligomers to the matrix.

In another embodiment, the present invention provides a method of enriching a sample of oligomer reactive antibodies comprising providing an affinity purification matrix as described above; loading the matrix with a sample comprising oligomer reactive antibodies; and isolating the oligomer reactive antibodies. The sample may be a biological fluid, such as IGIV, blood, serum, plasma, saliva, urine, or peritoneal fluid.

Additionally, the present invention provides an enriched sample of oligomer reactive antibodies. The antibodies may be enriched for binding to oligomers by 10 to 20 fold. The antibodies may be enriched for binding by about 15 fold. The present invention also provides a composition comprising oligomer reactive antibodies and a carrier. In one embodiment the composition may be a pharmaceutical composition and the carrier may be a pharmaceutically acceptable carrier. The carrier may be an adjuvant. The present invention also provides vaccines comprising oligomer reactive antibodies and a carrier. The vaccine may also contain an adjuvant.

The present invention also provides a method of generating oligomer reactive antibodies comprising using oligomers isolated by the method of the present invention as an immunogen.

In one aspect, the present invention provides a method of treating an amyloid disorder comprising administering the oligomer reactive antibodies to a subject in need thereof to treat the amyloid disorder. The amyloid disorder may be Alzheimer's disease, AIAPP amyloidosis, ATTR amyloidosis, or AL amyloidosis.

In another aspect, the present invention provides a method of screening for oligomer antibody reactivity comprising incubating a biological sample with the oligomer reactive antibodies. The present invention also provides a method of diagnosing a subject with amyloid disorder comprising obtaining a biological sample from a subject, and incubating the sample with oligomer reactive antibodies. The present invention also provides a method of using oligomer reactive antibodies to screen for the presence of antibodies in a patient that are reactive against amyloid assemblies. The biological sample may be bodily fluid such as blood, serum, or plasma from a patient. The biological sample may be tissue from a patient.

Oligomer reactive antibodies may be generated using cross-linked oligomers prepared by the method of the present invention and may be any antibody that binds amyloidogenic oligomers. The oligomer reactive antibodies of the present invention may be antibodies that bind AO oligomers.

### BRIEF DESCRIPTION OF TH DRAWINGS

FIGS. 1A-1C show SDS PAGE and Western blot analyses of soluble cross-linked Aβ40 oligomers, also known as soluble cross-linked β-amyloid protein species (CAPS), prepared from monomeric Aβ using HRP. SDS PAGE analysis of the dose-dependent effect of HRP (FIG. 1A) and H₂O₂ (FIG. 1B) on Aβ oligomer formation was monitored after a 1-day incubation at 37° C. using 4-12% Bis-Tris gels. Each reaction was carried out in PBS containing ˜14 μM Aβ, 250-650 μM H₂O₂ (250 μM for reactions in Panel A) and 0-2.2 μM HRP (1.1 μM HRP for reactions in Panel B). FIG. 1C shows Aβ Western blot analysis of Aβ oligomers prepared using 0-2.2 μM HRP and H₂O₂ as shown by Moir, R. D. et al. (2005) J. Biol. Chem., 280, 17458-17463.

FIG. 2 shows SDS PAGE analysis of insoluble CAPS prepared from monomeric Aβ40 using Cu²⁺. The dose-dependent effect of Cu²⁺ on Aβ oligomer formation was monitored by SDS PAGE after 1, 2, and 4 days incubation at 37° C. using 4-12% Bis Tris gels, respectively. Each reaction was carried out in PBS containing, 14 μM Aβ, 250 μM H₂O₂ and 0-25 mM CuSO₄. The last panel shows Aβ Western blot analysis of Aβ by Atwood, C. S. et al. (2004) Biochemistry, 43, 560-568, using 25 μM CuSO₄ and 250 μM H₂O₂ in PBS.

FIGS. 3A-3C show SDS PAGE and ThT fluorescence comparison of CAPS prepared from monomeric Aβ peptide using Cu²⁺ or HRP. SDS PAGE analysis of the dose dependent effect of HRP (FIG. 3A) and CuSO₄ (FIG. 3B) on Aβ oligomer formation was monitored after a 2-day incubation at 37° C. using 4-12% Bis Tris gels. Each reaction was carried out with 250 μM H₂O₂, as described in FIGS. 1 and 2. FIG. 3C shows a comparison of the ThT fluorescence of the reaction products with that of Aβ fibrils.

FIG. 4 shows SDS PAGE analysis of insoluble CAPS prepared by Cu²⁺ catalysis of quiescent or agitated soluble Aβ, or Aβ fibrils. Each reaction was carried out for up to 2-days at 37° C. in PBS containing, ˜72 μM Aβ (soluble peptide was prepared by high pH treatment) or ˜30 μM Aβ fibrils, 250 μM H₂O₂ and 0-1000 μM CuSO₄. The samples were run on 4-12% Bis-Tris gels.

FIG. 5 shows attempts to purify soluble CAPS using size exclusion gel chromatography column. ˜100 μg of Aβ oligomer reaction mix, prepared using ˜30 μM Aβ, 2.2 μM HRP and 250 μM H₂O₂, was loaded on to a 10 ml Superdex 75 or a Sephacryl S200 (GE Healthcare) column and 1 ml fractions collected. The amount of protein in each fraction was determined using the Micro-BCA assay (Pierce).

FIG. 6 shows reverse-phase HPLC trace of CAPS reaction product obtained using HRP. ˜10 μg of the Aβ aggregate reaction mix was injected onto a C3 Zorbax Column (Agilent) that was developed with a gradient of acetonitrile in aqueous 0.05% trifluoroacetic acid. Aβ40 oligomers were prepared as described in Materials and Methods in Example 1.

FIGS. 7A-7B show SDS PAGE analysis of HRP-bead catalyzed CAPS reaction products. FIG. 7A shows the effect of the amount of HRP-beads and incubation time on Aβ oligomer formation. FIG. 7B shows the effect of pre-blocking HRP-beads with various blocking agents on the amount of soluble oligomer product. Each redox reaction was carried out with gentle mixing with 20 μM Aβ, 250 μM H₂O₂ in PBS at 37° C. as described in Materials and Methods in Example 1. Samples loaded on to the gel were centrifuged to remove HRP-beads.

FIG. 8 shows a determination of an optimal reagent for disrupting HRP—CAPS interactions. Soluble Aβ oligomer reaction product, containing bound HRP, was precipitated by 1 mM CuSO₄ and the precipitant pelleted and the supernatant (sup.) removed. Various reagents were added to the pellet and the sample centrifuged to determine by SDS PAGE their ability to remove HRP. The pellet (pell.) was solubilzed by the addition of 5 mM EDTA in PBS before being loaded onto the gel. The oligomer reaction was formed with 1.1 μM HRP, as described in FIG. 5. 100 mM glycine buffer was at pH 10.5, and the gentle Ag/Ab Elution Buffer (Pierce) contained a high salt proprietary composition.

FIG. 9 shows a determination of the optimal guanidine-HCl concentration for purifying Cu²⁺ precipitated CAPS. SDS PAGE analysis shows that 3M guanidine-HCl is the optimal denaturant concentration for obtaining pure Aβ oligomers in a high yield (˜90%). Aβ oligomer formation was carried out by HRP catalysis as is described in FIG. 5.

FIGS. 10A-10B show SDS PAGE and ThT fluorescence analyses of purified cross-linked CAPS (prepared using peptide that was solubilized by high pH pretreatment). FIG. 10A shows SDS PAGE analysis of 3M guanidine-HCl treated CuSO₄ precipitated Aβ oligomers. The redox reactions were carried out with 1.1 μM HRP as described in FIG. 5. FIG. 10B shows relative ThT fluorescence of Aβ oligomer preparations compared with Aβ fibrils.

FIGS. 11A-11D show SDS PAGE analyses of purified CAPS, which were generated using Aβ40 or Aβ42, and HRP as the catalyst. Coomassie (FIG. 11A) and silver (FIG. 11B) stained SDS PAGE gel analysis of purified Aβ42 oligomer reaction product. FIG. 11C shows SDS PAGE analysis of purified Aβ40 oligomers. FIG. 11D shows a comparison of the molecular weights of oligomer products obtained using Aβ40 and Aβ42 reaction substrates. The Aβ40 and Aβ42 reactions were carried out in PBS containing ˜69 and ˜8 μM peptide, respectively, and 250 μM H₂O₂ and 1.1 μM HRP, as described in Materials and Methods in Example 1. Gdn. Sup. and Gdn. Pell. are abbreviations for guanidine-HCl supernatant and guanidine-HCl pellet, respectively.

FIGS. 12A-12B show Western blot analysis of Aβ and HRP in purified CAPS reaction samples. FIG. 12A shows anti-Aβ staining using commercial antibodies directed against the N-, C-, and mid portion of the peptide. Aβ oligomer purification by sequential treatment with CuSO₄ and EDTA did not alter the size distribution or solubility of the ultra-centrifuged oligomer product. FIG. 12B shows anti-HRP staining using a commercial antibody that shows there is no HRP present in the purified Aβ oligomer preparations.

FIG. 13 shows a schematic of the optimal protocol for preparing and purifying CAPS. Any oligomer pellet that still remains after the above treatment can be readily resolubilized by the addition of a high pH buffer (200 mM glycine, PBS and 5 mM EDTA, pH 10.5).

FIG. 14 shows electrospray ionization mass spectral analysis of purified CAPS. 4 μg of aggregates was loaded onto a C₈ reverse phase HPLC column and the eluent directed into the ion-spray of a single quadrupole mass spectrometer and the masses determined.

FIG. 15 shows the dityrosine fluorescence emission spectrum of purified CAPS. Wavelength spectra of 50 μM purified Aβ aggregates or Aβ40 monomer were determined with excitation at 320 nm. The larger maximum fluorescent signal obtained for Aβ42 oligomers is at least partly due to a larger emission band silt (8 nm compared) compared to that for Aβ40 experiments (4 nm).

FIGS. 16A-16F show electron micrographs of purified CAPS. The micrographs show typical globular and protofibril-like Aβ aggregates. Aβ42 (FIGS. 16A, 16C, 16E) and Aβ40 (FIGS. 16B, 16D, 16F) aggregates were negatively stained with 0.5% uranyl acetate. The large bar is the scale for FIGS. 16E and 16F.

FIGS. 17A-17B show Western blot analysis of Aβ and HRP in purified CAPS reaction samples. FIG. 17A shows anti-Aβ staining using commercial antibodies directed against the N-, C-, and mid portion of the peptide. Aβ oligomer purification by sequential treatment with CuSO₄ and EDTA did not alter the size distribution or solubility of the ultra-centrifuged oligomer product. FIG. 17B shows anti-HRP staining using a commercial antibody that shows there is no detectable HRP in the purified aggregate preparation (rxn2+EDTA).

FIG. 18 shows binding curves for enriched anti-fibril IGIV binding to Aβ40 monomer, CAPS and fibrils. , ▪, ∘, data symbols for anti-fibril antibodies binding to cross-linked Aβ oligomers, fibrils, and monomer, respectively.

FIG. 19 shows determination of anti-CAPS reactivity of IgGs contained in (normal) human plasma samples. Results of EuLISA using a 1:20 dilution of plasma samples and plate-immobilized CAPS, which were prepared using the Aβ40 peptide. The signal on each plate was normalized using a standard curve determined from control plasma samples. The plasma number designations were provided by Baxter Bioscience.

FIG. 20 shows comparison of anti-Aβ40 fibril and CAPS reactivity's of IgGs contained in (normal) human plasma samples. Results of EuLISA for fibril (black bars) and oligomer (red bars) fibrils, respectively.

FIG. 21 shows comparison of anti-Aβ 40 fibril and monomer reactivity's of IgGs contained in (normal) human plasma samples. Results of EuLISA for fibril (black bars) and monomer (red bars) fibrils, respectively.

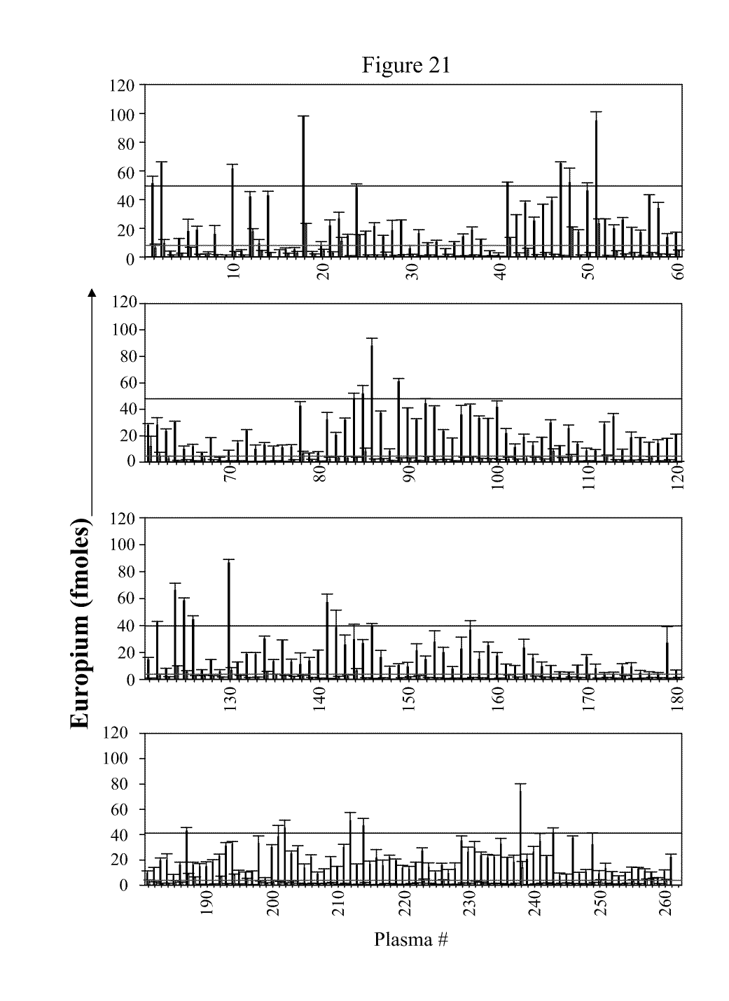

FIGS. 22A-22E show affinity-purified human immune globulin contains LC fibril- and Aβ conformer-reactive antibodies. Antibody titration curves for affinity purified (closed circles), unfractionated (open circles), and residual (closed triangles) IgG against the conformer used for affinity purification: LC fibrils (FIG. 22A); Aβ40 fibrils (FIG. 22B); CAPS (FIG. 22C); Aβ40 monomer (FIG. 22D); and equimolar mixture of N- and C-terminal cysteinylated F19P Aβ40 monomer peptides (FIG. 22E). Binding studies were carried out using 400 ng plate-immobilized antigen and the amount of bound IgG was quantitated by europium time-resolved fluorescence. The values shown represent the mean SD of triplicate analyses.

FIGS. 23A-23F show Aβ conformer cross-reactivity of LC fibril- and Aβ40 conformer affinity-purified and unfractionated human immune globulin. Antibody titration curves for affinity purified and unfractionated IGIV preparations against plate-immobilized Aβ40 conformers-fibrils (closed circles), CAPS (closed triangles), wild-type (open circles), and F19P (open triangles) monomer. IGIV was affinity purified against LC fibrils (FIG. 23A), Aβ40 fibrils (FIG. 23B), CAPS (FIG. 23C), Aβ40 monomer (FIG. 23D), and an equimolar mixture of N- and C-terminal cysteinylated F19P Aβ40 monomer (FIG. 23E. Unfractionated IGIV is seen in FIG. 23F.

FIG. 24 shows affinity column depletion of LC fibril- and Aβ conformer-reactive antibodies contained in human immune globulin. Comparison of the amount of LC fibril and Aβ conformer-reactive antibodies isolated from one passage of unfractionated (closed bars) with the amount of reactivity obtained with residual IGIV preparations, which was prepared by passing 10-20 mg/ml IGIV 3-4 times through a LC fibril (vertically lined bars), CAPS (grey bars), or Aβ40 monomer (open bars) affinity column. Each column contained ˜1-3 mg/ml of an amyloidogenic conformer. The percentage of antigen-reactive antibody was determined spectrophometrically.

FIGS. 25A-25B show a comparison of the reactivity of Aβ40 monomer column purified antibody against Aβ40 fibrils, CAPS, and monomer. FIG. 25A shows competition binding studies involving intact Aβ40 monomer affinity-purified IGIV versus a commercially-derived N-terminal-reactive anti-Aβ Ab (MAB 1560; Chemicon, Temecula, Calif.) in the absence (closed bars) or presence of a 100-fold molar excess of wild-type (open bars) or F19P (grey bars) Aβ40 monomer, against Aβ40 monomer coated directly or plate-immobilized using poly-L-lysine/glutaraldehyde. FIG. 25B shows competition binding studies involving Aβ40 monomer purified antibody F(ab′) fragment binding with Aβ40 monomer in the presence or absence of wild-type or F19P Aβ40 monomer, CAPS, or Aβ fibrils

FIGS. 26A-26F show Aβ oligomer-reactivity of Aβ40 fibril and CAPS-isolated human immune globulin. Binding of CAPS-purified (FIG. 26A) and Aβ fibril-isolated (FIG. 26B) IgGs to plate-immobilized Aβ40 CAPS in the presence or absence of a 50-fold molar excess of competitors (see x-axis labels). Mod. ovalb. agg. stands for reduced and alkylated ovalbumin aggregates. Western blot analysis of Aβ40 CAPS binding by IgGs in IGIV purified by CAPS (FIG. 26C), Aβ40 fibrils (FIG. 26D), a commercially derived N-terminal Aβ-reactive mAb (MAB1560; Chemicon, Temecula, Calif.) (FIG. 26E). FIG. 26F shows a Commassie-stained 4-12% bis-tris SDS gel. Fifty-100 nM of Aβ40 oligomer purified antibody was used in the microtiter plate and Western blot experiments.

FIGS. 27A-27F show the effect of human plasma on Aβ conformer-reactivity of Aβ40 fibril- and CAPS-isolated human immune globulin. Antibody binding was carried out in the absence (open circles) or in the presence of a human plasma (closed circles), or with plasma alone (closed squares). FIGS. 27 A, C, and E show anti-fibril enriched immune globulin binding to Aβ40 fibrils, CAPS, and monomer, respectively. FIGS. 27B, D, and F show anti-CAPS enriched immune globulin binding to Aβ fibrils, CAPS, and monomer, respectively. Human plasma was added to stock antibodies (˜0.2 mg/ml) at 1:10 dilution.

FIG. 28 shows a schematic of Aβ-reactivity of Aβ conformer affinity purified IGIV. The bar charts reflect antibody binding that was carried out at ˜100 nM. The designation of an antibody as either anti-fibril- or CAPS-reactive reflects its preferential binding to the particular species, although each of these antibodies can still cross-react with fibrils and CAPS. Reactivity against plate or column-immobilized Aβ monomer is not against the peptide per se, but conformational epitope(s) that is induced by immobilization.

### DETAILED DESCRIPTION

### A. General Description

The present invention provides a methodology for obtaining a requisite amount of purified cross-linked redox modified oligomers as material for affinity chromatography, active vaccination, and substrate to determine whether there are oligomer reactive antibodies in IGIV or donor human plasma samples. The cross-linked oligomers may serve as an antigen for affinity isolation of anti-oligomer reactive antibodies; immunogen for generating oligomer antibodies; and material for characterizing oligomer reactive antibodies. The identification, production and characterization of oligomer reactive antibodies have therapeutic potential given that scientists believe oligomers are pathogenic species.

As an example, the present invention shows that purified dityrosine cross-linked oligomers, for example Aβ oligomers, also termed soluble cross-linked β-amyloid protein species (CAPS), are an excellent source for affinity chromatography isolation, production and characterization of Aβ oligomer-reactive antibodies. The present invention also shows that naturally occurring human antibodies against Aβ oligomers in immune globulin intravenous (IGIV), which were isolated by affinity chromatography, cross-react with Aβ fibrils. These antibodies bind to common fibril-related conformational epitope(s) on fibrils and oligomers.

The present invention is also based in part on the finding that human sera contain antibodies that bind to common conformational epitopes on CAPS, Aβ fibrils, and LC fibrils, with EC₅₀ values of ˜40 nM. Little, if any, binding occurred with Aβ monomers or SDS-stable oligomers as well as with lysozyme oligomers or non-amyloidogenic ovalbumin aggregates. Affinity chromatography, using LC fibrils, Aβ fibrils, CAPS, or wild-type and F19P monomers as well as competition binding studies, confirmed that Aβ conformer-reactivity was directed against a limited number of conformational epitopes on the aggregated peptide, with negligible binding to the monomeric peptide. Antibodies eluted off CAPS and fibril columns bound to common epitopes on CAPS and fibrils, with preferential reactivity against the conformer used for isolation. In the presence of human plasma, CAPS isolated antibodies retained more activity against aggregated Aβ than fibril-purified IgGs, indicating that these antibody preparations contained diverse IgG populations.

### B. Definitions

As used herein, a “diagnostic agent” or “imaging agent” refers to agents including those that are pharmaceutically acceptable agents that can be used to localize or visualize amyloid deposits by various methods.

As used herein, “fragments” of oligomer reactive antibodies include but are not limited to Fc, Fab, Fab′, F(ab′)₂ and single chain immunoglobulins.

As used herein, “gamma globulin”, is the serum globulin fraction that is mainly composed of IgG molecules.

As used herein, “IGIV” “IVIG” or “intravenous immunoglobulins” refers to gamma globulin preparations suitable for intravenous use, such as those IGIV preparations which are commercially available. IGIV may also be isolated from the blood of donors and are suitable for intravenous administration. IGIV can be isolated from different mammals, including non-human sources, such as mouse, rat, hamster, guinea pig, dog, cat, rabbit, pig, goat, sheep, cow, chimpanzee, and monkey. In one embodiment of the invention, human IGIV preparations are used for intravenous administration. Human IGIV preparations are available from various commercial sources. The commercially available IGIV preparations contain mainly IgG molecules.

As used herein, the term an “immunologically effective amount” means that the administration of that amount to a subject, either in a single dose or as part of a series, is effective for treatment of amyloidosis. This amount varies depending upon the health and physical condition of the subject to be treated, the species of the subject to be treated (e.g. non-human mammal, primate, etc.), the capacity of the subject's immune system to synthesize antibodies, the degree of protection desired, the formulation of the vaccine and other relevant factors. It is expected that the amount will fall in a relatively broad range that can be determined through routine trials.

As used herein, the term “oligomer” refers to covalent and non-covalent dimer or higher aggregates of amyloidogenic proteins or peptides that are on or off pathway assembly intermediates of fibril formation. Examples of such oligomers include but are not limited to annular, spherical/globular oligomers, CAPS, and amyloid derived diffusible ligands (ADDLS).

As used herein, the phrase “specifically (or selectively) binds to” or “specifically (or selectively) immunoreactive with” refers to a binding reaction which is determinative of the presence of the molecule of interest in the presence of a heterogeneous population of proteins and other biologics. Thus, under designated assay conditions, the specified ligands (e.g., an antibody) bind to a particular molecule (e.g., an epitope on cross-linked Aβ oligomers) and do not bind in a significant amount to other molecules present in the sample. In affinity purification, the ligand may be the cross-linked Aβ oligomers conjugated to an affinity purification matrix and the molecule of interest is the cross-linked Aβ oligomer reactive antibodies being enriched for binding amyloidogenic oligomers.

As used herein, “pharmaceutical composition” or “formulation” refers to a composition comprising an agent or compound together with a pharmaceutically acceptable carrier or diluent. A pharmaceutically acceptable carrier includes, but is not limited to, physiological saline, ringers, phosphate buffered saline, and other carriers known in the art. Pharmaceutical compositions may also include stabilizers, anti-oxidants, colorants, and diluents. Pharmaceutically acceptable carriers and additives are chosen such that side effects from the pharmaceutical agent are minimized and the performance of the agent is not canceled or inhibited to such an extent that treatment is ineffective.

As used herein, a sample, or biological sample may refer to a collection of fluid and or cellular material derived from a subject. The sample may be derived from tissue. The sample may be derived from a biological fluid. Examples of tissue include bone and muscle and may be derived from any organ of the body, such as the brain, heart, liver, kidney, lung, intestine, stomach, gonads, circulatory system, spinal cord, pancreas, adrenal gland, bladder, prostate, skin, spleen, and colon. Biological fluids may include, for example, blood, sputum, saliva, semen, vaginal fluid, excrement (such as urine and feces), cerebrospinal fluid, gastric acid, interstitial fluid, and bile.

As used herein, “subject” can be a human, a mammal, or an animal. The subject being treated is a patient in need of treatment.

As used herein, “therapeutically effective amount” refers to that amount of the agent or compound which, when administered to a subject in need thereof, is sufficient to effect treatment. The amount of antibodies such as cross-linked Aβ oligomer reactive antibodies which constitutes a “therapeutically effective amount” will vary depending on the severity of the condition or disease, and the age and body weight of the subject to be treated, but can be determined routinely by one of ordinary skill in the art having regard to his/her own knowledge and to this disclosure.

As used herein, the term “treatment” includes the application or administration of a therapeutic agent to a subject or to an isolated tissue or cell line from a subject, who is afflicted with amyloidosis, a symptom of amyloidosis or a predisposition toward amyloidosis, with the goal of curing, healing, alleviating, relieving, altering, remedying, ameliorating, improving or affecting the disease, the symptoms of disease or the predisposition toward disease.

### C. Specific Embodiments

Oligomers in Amyloidosis

Amyloidosis is a group of progressive, incurable, metabolic diseases in which protein is deposited in specific organs (localized amyloidosis) or throughout the body (systemic amyloidosis). Amyloid proteins are manufactured by malfunctioning bone marrow and elsewhere in the body. The accumulation of amyloid deposits impair normal body function causing organ failure or death.

Alzheimer's disease (AD) is the most common, of over 25, incurable misfolding diseases that are termed the amyloidoses (Merlini et al., 2003; Westermark et al., 2005; Stefani, 2004; Monaco et al., 2006; Chiti et al., 2006; Golde, 2005; Hardy et al., 2002; Goedert et al., 2006). Each disorder involves the abnormal aggregation of self-protein of diverse chemical composition that ultimately results in deposition as amyloid fibrils in the brain or other vital organs, leading to organ failure and eventually death (Merlini et al., 2003; Westermark et al., 2005; Stefani, 2004; Monaco et al., 2006; Chiti et al., 2006; Golde, 2005; Hardy et al., 2002; Goedert et al., 2006). The hallmark of AD is the abnormal processing of β-amyloid protein (Aβ), a proteolyzed transmembrane fragment of amyloid precursor protein (APP), which exists in the cerebrospinal fluid as soluble monomers and oligomers, and it eventually deposits as amyloid fibril in neuritic plaques (Stefani, 2004; Chiti et al., 2006; Goedert et al., 2006;).

Tyrosine cross-linking has been proposed as a mechanism of Aβ oligomerization in vivo, since tyrosine residues in synthetic human Aβ can be cross-linked by peroxidase-catalyzed oxidation systems (Galeazzi et al., 1999). As Rat Aβ, unlike human Aβ, lacks a tyrosine residue (Atwood et al., 1997), it is therefore resistant to metal-catalyzed oxidative oligomerization, and this perhaps explains the rarity of amyloid deposits in these animals (Vaughan and Peters, 1981).

The oxidative processes which give rise to covalent cross-linking of proteins via tyrosine are also associated with other disorders which are characterised by pathological aggregation and accumulation of specific proteins. Thus, tyrosine cross-linking may also be important in other neurodegenerative diseases such as Parkinson's disease, and other conditions in which α-synuclein fibrils are deposited. These include Parkinson's disease itself, dementia with Lewy body formation, multiple system atrophy, Hallerboden-Spatz disease, and diffuse Lewy body disease. Exposure of recombinant α-synuclein to nitrating agents results in nitration of tyrosine residues as well as oxidation of tyrosine to form DT; this results in cross-linking of α-synuclein to form stable aggregates (Souza et al, 2000). It was also reported that monoclonal antibodies raised against nitrated synuclein bound specifically to Lewy bodies and to glial cell inclusions in a variety of synucleinopathies (Duda et al., in preparation referred to in Souza et al., 2000).

Published Application 20040013680 discloses a method of prophylaxis, treatment or alleviation of a condition characterized by pathological aggregation and accumulation of a specific protein associated with an immunizing-effective dose of one or more tyrosine cross-linked compounds, and optionally also comprising copper ions complexed to the compound. Alternatively passive immunization against a tyrosine cross-linked compound also may be used.

Purification and Characterization of CAPS

The present invention is based in part on the discovery of a novel method of preparing, isolating, and/or purifying antibodies directed against amyloidogenic oligomers. The present invention provides a reproducible method of isolating and/or purifying antibodies to oligomers. As an example, the present invention provides a method of obtaining purified antibodies that specifically bind to CAPS. The latter cross-linked Aβ oligomers have been shown to be neurotoxic.

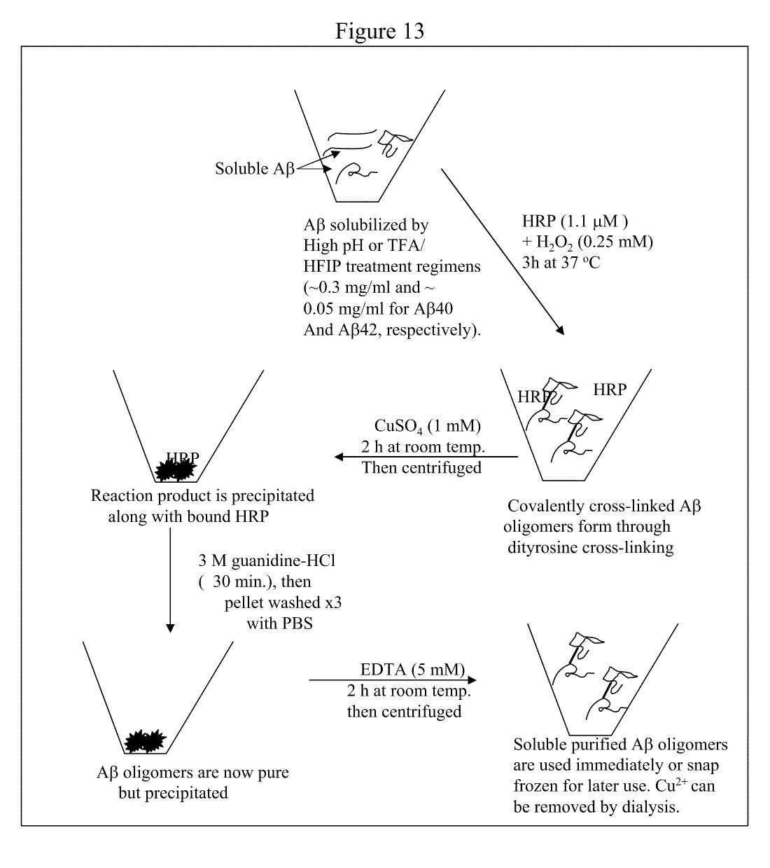

FIG. 13 summarizes the steps in the preparation and purification of CAPS. The method comprises catalyzing the formation of cross-linked Aβ oligomers from Aβ peptides, and precipitating the CAPS to obtain highly purified cross-linked Aβ aggregates. These steps could also be used to prepare and, or purify dityrosine cross-linked amyloidogenic oligomers from synthetic or patient-derived amyloidogenic proteins or peptides.

In one embodiment, the Aβ peptides may be solubilized prior to cross-linking by high pH treatment, such as by dissolving the Aβ peptides in sodium hydroxide. In another embodiment, the Aβ peptides may be solubilized by sequential treatments using trifluoroacetic acid (TFA) and 1,1,1,3,3,3-hexafluoro-2-propanol (HFIP). Aβ peptides may be solubilized by other methods and agents well-known in the art. Other reagents that may be useful in solubilizing Aβ peptides include potassium hydroxide, ammonium hydroxide, and dimethyl sulfoxide. The Aβ peptides may also be solubilized directly into distilled water, and buffer such as PBS would be added.

The Aβ peptides may be induced to form CAPS by treatment with a catalyst such as horseradish peroxidase (HRP) or Cu²⁺ ions. Cu²⁺ ions may be added in the form of CuSO₄ or CuCl₂. Other appropriate redox reagents may also be used to induce cross-linked oligomers. The catalyst may be conjugated to a matrix. For example, HRP may be conjugated to beads and the Aβ peptides are added to the HRP-beads. The HRP-beads may be treated with a blocking agent prior to incubating with the Aβ peptides. Blocking agents include but are not limited to bovine serum albumin (BSA), gelatin, and other appropriate blocking agents.

Alternatively, to increase the efficiency of dityrosine cross-linking using copper as the catalyst, a more aggregated Aβ peptide may be used as the substrate with the catalyst.

The method further comprises precipitating the CAPS by adding Cu²⁺ ions in the form of copper sulfate. The inventors unexpectedly discovered that precipitating the cross-linked Aβ oligomers with Cu²⁺ ions resulted in highly purified cross-linked Aβ oligomers, since Cu²⁺ precipitation resulted in the removal of about 80% of the HRP and oligomer yield was greater than 90%, the highest of any purification method used. The inventors also discovered that HRP is more efficient than copper in producing oligomers.

The method may further comprise incubating the precipitated CAPS under conditions allowing removal of residual catalyst (such as HRP) and Cu²⁺ ions. Agents and/or conditions that disrupts the protein-protein interactions are effective in removing residual catalyst and Cu²⁺ ions. Such agents or conditions include but are not limited to guanidine hydrochloride, SDS, urea, high salt concentration, extreme pH. Guanidine hydrochloride is the preferred reagent because it is relatively inert.

The method may further comprise washing CAPS in buffer, such as PBS, to remove guanidine hydrochloride. The oligomers may then be resolubilized in buffer containing EDTA and centrifuged at about 20,000 g to remove residual impurities from the supernatant containing the purified cross-linked Aβ oligomers. Any oligomer pellet may be resolubilized by the addition of a high pH buffer, such as 200 mM glycine, PBS and 5 mM EDTA, pH 10.5.

The purified soluble CAPS may be used immediately or snap frozen for later use. The residual Cu²⁺ may be removed by dialysis.

After purification, the oligomers may be characterized by biophysical methods. Such methods include but are not limited to electrospray ionization mass spectrometry, dityrosine fluorescence, electron microscopy, thioflavin T fluorescence, Western blot analysis, and binding to antibodies, i.e. anti-Aβ antibodies (enriched anti-fibril IGIV or commercial antibodies).

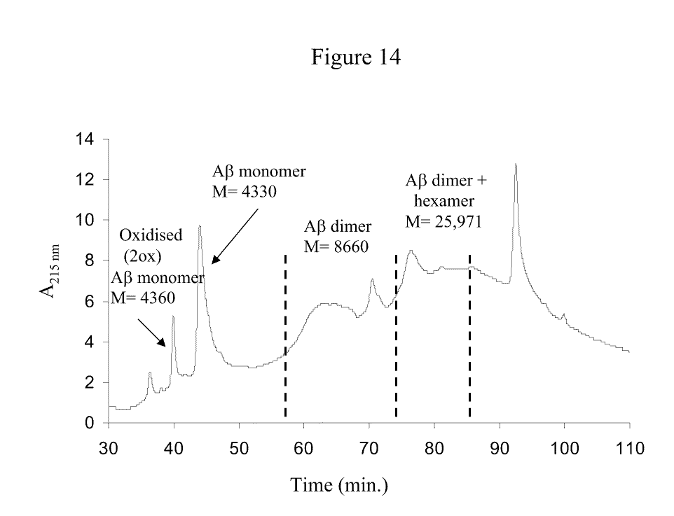

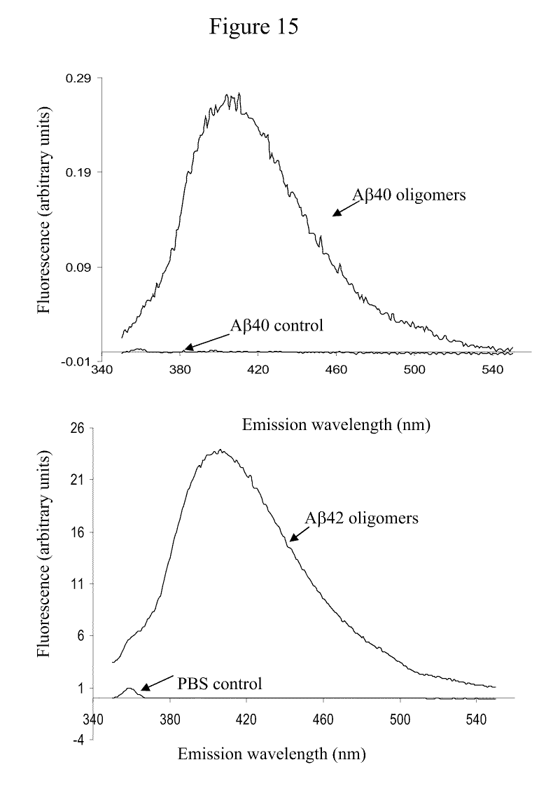

Biophysical characterization of CAPS indicated that these aggregates consisted of globular and protofibril-like assemblies that typify fibril assembly intermediates. The inventors confirmed via electrospray ionization mass spectral analysis that the cross-linked Aβ oligomers contained covalently cross-linked Aβ dimers and hexamers (FIG. 14). Presumably, these are cross-linked through dityrosines (Galeazzi et al., 1999; Atwood et al., 2004; Ali et al., 2006) as purified Aβ oligomers gave typical dityrosine fluorescence emission wavelength spectra with an emission maximum at ˜418 nm by excitating at 320 nm (FIG. 15). In contrast, monomeric Aβ controls did not fluoresce at these wave-lengths (FIG. 15). Mass spectral analyses also confirmed that cross-linked Aβ molecules were of a molecular weight consistent with the unmodified peptide, covalently bound by dityrosine; further, no gross redox modification of the aggregated peptide was evidenced. However, due to low ionization of the peptide and the heterogenous nature of the oligomeric sample, it was not possible to determine if all Aβ oligomers (including trimers, and tetramers that were observed by SDS PAGE) were covalently cross-linked and unmodified.

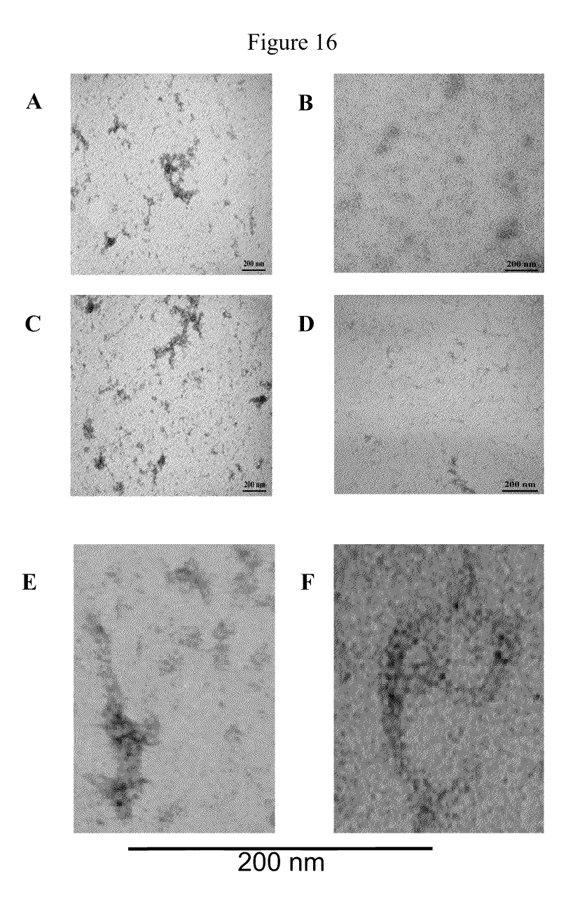

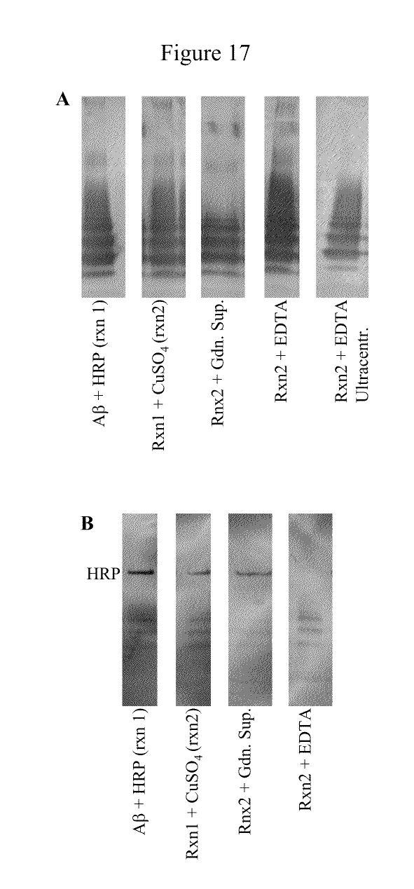

Electron micrographs of purified CAPS showed that these molecules were globular and consisted of protofibril-like aggregates that were much larger than that observed by SDS PAGE and typified Aβ fibril assembly intermediates (FIG. 16). Additionally, Western blot analyses using a mixture of 3 commercial antibodies that each recognize an epitope in the N-terminal, C-terminal or mid portion of the Aβ peptide showed that oligomer preparations contained SDS-stable high molecular weight oligomers (>tetramers) that, presumably, were not at a high enough concentration to be detected by SDS PAGE (FIG. 17).

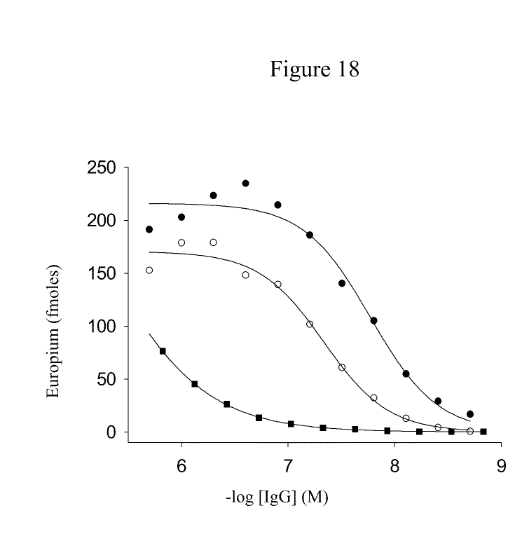

To determine whether the purified CAPS contain amyloid fibril-like epitopes, EuLISA antibody binding curves were constructed using enriched anti-fibril IGIV against, Aβ fibrils, oligomers, and monomer. FIG. 18 shows that anti-fibril enriched IGIV has similar affinity for Aβ oligomers and fibrils (EC₅₀ values of ˜30 nM), but notably weaker binding to Aβ monomer (Ec₅₀ values of ˜1 μM). Taken together, these results are indicative of fibril-associated epitope(s) on purified cross-linked Aβ oligomers.

The present invention provides a method of obtaining highly purified oligomers containing amyloid fibril-like epitopes. As an example, the inventors have purified CAPS using the new method. The method is applicable to the purification of other amyloidogenic oligomers. The oligomers may be from naturally occurring sources, prepared by recombinant means, or from synthetic sources. See Table 1 for a list of peptides that may be used in the method of the present invention to prepare cross-linked oligomers. These peptides may also comprise one or more tyrosine residues.

Uses of Purified Oligomers

The purified oligomers prepared by the method of the present invention may be used in various ways. In one embodiment, the purified oligomers may be used to isolate and/or purify oligomer reactive antibodies or fragments thereof from biological fluids. In another embodiment, the purified oligomers may be used to screen for and detect oligomer reactive antibodies or fragments thereof in a biological sample. The purified oligomers may be used as a ligand in these methods. The oligomers may also be used as an immunogen to generate oligomer reactive antibodies.

Biological sample may include tissues, cells, extracellular matrix, and biological fluids. Biological fluids include but are not limited to blood, plasma, serum, cerebrospinal fluid, urine, peritoneal fluid, and saliva.

Oligomer Reactive Antibodies

The present invention provides oligomer reactive antibodies, for instance AU oligomer reactive antibodies, generated using CAPS prepared by the methods described above as immunogen. The oligomer reactive antibodies of the present invention may be isolated and/or purified by an affinity purification process using oligomers prepared by the method described above as ligand. The methods may use a biological sample obtained from a subject, such as a sample of tissue or fluid derived from the subject.

The present invention also provides cross-linked oligomer reactive antibodies as a whole molecule or fragments thereof such as the F(ab′)₂ or Fc fragment by itself in treating subjects. Prior to administration, the antibody preparation of the present invention may be subject to treatment such as enzymatic digestion (e.g. with pepsin, papain, plasmin, glycosidases, nucleases, etc.), heating, etc. and/or further fractionated but will normally be used as commercially available. Thus, administered compositions may comprise primarily intact antibody, antibody fragments, or mixtures thereof. Hence, by antibody fragments of the present invention is meant preparations of oligomer reactive antibody fragments suitable for in vivo administration.

In one embodiment, the oligomer reactive antibodies or fragments thereof of the present invention are enriched for binding to amyloidogenic oligomers and to partially denatured amyloidogenic precursor polypeptides, especially when plate adsorbed. They can be used to treat subjects suffering from amyloidosis. The oligomer reactive antibodies and fragments thereof of the present invention may be used to neutralize the cytotoxic effect of oligomers in subjects in need thereof. Generally, oligomers are more cytotoxic than fibrils. Accordingly, the oligomer reactive antibodies play a role in clearing the soluble pool of oligomers and provide beneficial effect in patients suffering from amyloidosis. The oligomer reactive antibodies may also be used to detect amyloid deposits in subjects.

Monoclonal and polyclonal antibodies of the present invention can be obtained by immunizing animals with oligomers prepared by the methods described above or other molecules that mimic the oligomer epitopes in amyloid deposits. These antibodies will bind epitopes on amyloid deposits and soluble oligomers.

Polyclonal antibodies that bind oligomers can be prepared by any methods known in the art. As described, polyclonal antibodies may be prepared by immunizing a suitable subject with cross-linked oligomers prepared by the method of the present invention or polypeptides, peptides or molecules that mimic the oligomer epitopes of amyloid deposits. The desired polyclonal antibodies may be isolated from the sera of the subject. In one embodiment, the polyclonal antibody compositions are ones that have been selected for antibodies that recognize or bind specifically to amyloidogenic oligomers.

Monoclonal antibodies that bind amyloidogenic oligomers may be made by the hybridoma method first described by Kohler et al, 1975, or may be made by recombinant DNA methods (see, e.g., U.S. Pat. No. 4,816,567, which is herein incorporated by reference in its entirety). Monoclonal antibodies may also be isolated from phage antibody libraries using the techniques described in Clackson et al., 1991 and Marks et al., 1991, for example.

The phrase “specifically (or selectively) binds” to an antibody or “specifically (or selectively) immunoreactive with”, refers to a binding reaction that is determinative of the presence of amyloidogenic oligomers in a heterogeneous population of proteins and other biologics. Thus, under designated immunoassay conditions, the specified antibodies bind amyloidogenic oligomers at least two times the background and do not substantially bind in a significant amount to other proteins or biologics present in the sample. A variety of immunoassay formats may be used to select antibodies specifically immunoreactive with a partially denatured amyloidogenic precursor proteins. For example, solid-phase ELISA immunoassays are routinely used to select antibodies specifically immunoreactive with a protein (see, e.g., Harlow & Lane, 1988, for a description of immunoassay formats and conditions that can be used to determine specific immunoreactivity). Typically a specific or selective reaction will be at least twice background signal or noise and more typically more than 10 to 100 times background.

The monoclonal antibodies of the present invention also include chimeric antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., 1984), such as binding to amyloid oligomers and to partially denatured amyloidogenic precursor proteins. A chimeric antibody is a molecule in which different portions are derived from different animal species, such as those having a variable region derived from a murine mAb and a human immunoglobulin constant region. Chimeric antibodies may be obtained by splicing the genes from a mouse antibody molecule of appropriate antigen specificity together with genes from a human antibody molecule of appropriate biological activity can be used (Morrison et al., 1984, Proc. Natl. Acad. Sci. USA, 81:6851 5; Neuberger et al., 1984, Nature, 312:604 8; Takeda et al., 1985, Nature, 314:452 4).

The present invention also includes humanized antibodies (see, e.g., U.S. Pat. No. 5,585,089 which is incorporated by reference in its entirety) that bind amyloidogenic oligomers. “Humanized” forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all, or substantially all, of the FRs are those of a human immunoglobulin sequence. The humanized antibody optionally also will comprise at least a portion of an immunoglobulin constant region (Fc), typically that of a human immunoglobulin. For further details, see Jones et al., 1986; Riechmann et al., 1988; and Presta, 1992).

Moreover, the present invention includes single chain antibodies (U.S. Pat. No. 4,946,778; Bird, 1988; Huston et al., 1988; Ward et al., 1989) that bind amyloidogenic oligomers and partially denatured amyloidogenic precursor proteins. Single chain antibodies are formed by linking the heavy and light chain fragments of the Fv region via an amino acid bridge, resulting in a single chain polypeptide.

The present invention also provides oligomer reactive antibodies and fragments thereof by recombinant means known in the art.

Affinity Purification

The present invention is based in part on cross-linked oligomers being an excellent source of material for affinity chromatography purification, production and characterization of oligomer reactive antibodies. As an example, the present invention shows that cross-linked Aβ oligomers can be used as a ligand for affinity purification of Aβ oligomer reactive antibodies.

In one aspect of the invention, the oligomers prepared by the method described above are used as ligands to isolate and or purify oligomer reactive antibodies or fragments thereof by affinity purification. The present invention provides an affinity purification matrix comprising oligomers prepared by the present invention and a method of preparing such an affinity purification matrix. The oligomers may be conjugated to an affinity purification matrix, such as sepharose.

Affinity purification (also called affinity chromatography) makes use of specific binding interactions between molecules. Affinity purification broadly refers to separation methods based on a relatively high binding capacity (“affinity”) of a target material to be purified, generally termed a “ligate”, for a complementary ligand. Affinity purifications can be accomplished in solution. However, more typically, a particular ligand is chemically immobilized or “coupled” to a solid support so that when a complex mixture is passed over the column, only those molecules having specific binding affinity to the ligand are purified. In the affinity purification method of the present invention, the ligand used for isolating oligomer reactive antibodies is the oligomers prepared by the method of the present invention.

Affinity purification generally involves the following steps:
1. Incubate crude sample with the immobilized ligand support material to allow the target molecule in the sample to bind to the immobilized ligand.
2. Wash away non-bound sample components from solid support.
3. Elute (dissociate and recover) the target molecule from the immobilized ligand by altering the buffer conditions so that the binding interaction no longer occurs.
   A single pass of a sample through an affinity column can achieve greater than 1,000 fold purification of a molecule from a crude mixture.

Affinity purification involves the separation of molecules in solution (mobile phase) based on differences in binding interaction with a ligand that is immobilized to a stationary material (solid phase). A support or matrix in affinity purification is any material to which a biospecific ligand may be covalently attached. Typically, the material to be used as an affinity matrix or resin is insoluble in the system in which the target molecule is found. Usually, but not always, the insoluble matrix is a solid. Hundreds of substances have been described and employed as affinity matrices.

Useful affinity supports are those that contain: a high surface area to volume ratio, chemical groups that are easily modified for covalent attachment of ligands, minimal nonspecific binding properties, good flow characteristics, and mechanical and chemical stability. Ideally, matrices for ligand immobilization should have a large surface area and comprise an open and loose porous network to maximize interaction of matrix-bound ligand with ligate (molecule of interest during the separation procedure). The matrix should be chemically and biologically inert, at the very least toward the ligand and ligate; be adapted for ligand immobilization; and be stable under reaction conditions employed, for example during matrix activation, ligand binding, and ligand-ligate complex formation, especially with respect to the solvent, pH, salt, and temperature employed. The matrix should also be stable for a reasonable length of time under ordinary storage conditions. To minimize competition for the target material and maximize purity of recovered product, supports for immobilization of ligands, especially biospecific ligands, should be free from extraneous ion exchange sites, and should not promote non-specific binding. Matrices, especially those used in pressurized affinity separation techniques, should be mechanically strong and be able to withstand at least the moderate pressures typical of these conventional systems (up to about 5 bar, for example). Matrices may be derivatized, for example, to promote ligand immobilization or to permit improved ligand target interaction.

There are a number of useful matrix materials such as agarose gels; cellulose; dextran; polyacrylamide; hydroxyalkylmethacrylate gels; polyacrylamide/agarose gels; ethylene copolymers, especially with polyvinyl acetate; copolymers of methacrylamide, methylene bis-methacrylamide, glycidyl-methacrylate and/or allyl-glycidyl-ether (such as Eupergit C, Rohm Pharma, Darmstadt, West Germany); and diol-bonded silica. The present invention provides amyloid oligomers which may be linked covalently to a matrix material, such as N-hydroxysuccinimide (HS)-activated Sepharose®4 fast-flow pre-activated agarose matrix.

Most commonly, ligands are immobilized or “coupled” directly to solid support material by formation of covalent chemical bonds between particular functional groups on the ligand (e.g., primary amines, sulfhydryls, carboxylic acids, aldehydes) and reactive groups on the support. However, other coupling approaches are also possible.

Most affinity purification procedures involving protein-ligand interactions use binding buffers at physiologic pH and ionic strength, such as phosphate buffered saline (PBS). For obvious reasons, this is especially true when antibody-antigen or native protein-protein interactions are the basis for the affinity purification. Once the binding interaction occurs, the support is washed with additional buffer to remove unbound components of the sample. Nonspecific (e.g., simple ionic) binding interactions can be minimized by adding low levels of detergent or by moderate adjustments to salt concentration in the binding and/or wash buffer. Finally, elution buffer is added to break the binding interaction and release the target molecule, which is then collected in its purified form. Elution buffer can dissociate binding partners by extremes of pH (low or high), high salt (ionic strength), the use of detergents or chaotropic agents that denature one or both of the molecules, removal of a binding factor or competition with a counter ligand. In most cases, subsequent dialysis or desalting may be used to exchange the purified protein from elution buffer into a more suitable buffer for storage or downstream analysis.

The most widely used elution buffer for affinity purification of proteins is about 0.1 M glycine.HCl, at about pH 2.5-3.0. This buffer effectively dissociates most protein-protein and antibody-antigen binding interactions without permanently affecting protein structure. However, some antibodies and proteins may be damaged by low pH, so eluted protein fractions should be neutralized immediately by collecting the eluting fractions in tubes containing 1/10th volume of alkaline buffer such as about 1 M Tris.HCl, at about pH 8.5 to 9.0. Other elution buffers for affinity purification of proteins are well known to a person of ordinary skill in the art.

Affinity purification may also be carried out in batch mode, for example in a beaker or a similar container. The ligand, oligomers prepared by the present invention, may be conjugated to an appropriate resin or matrix and placed in a beaker for affinity purification. A biological sample may be mixed and swirled with the resin to allow binding to the oligomers and washed in the beaker with buffers. Oligomer reactive antibodies that bind amyloidogenic oligomers may be eluted and isolated as described earlier.

The crude sample may be a biological sample, such as a fluid or tissue derived from a subject. Tissue samples may be lysed to extract sub-cellular material and to disrupt plamsa membrane integrity.

As an example, the present invention provides an affinity purification matrix comprising CAPS conjugated to sepharose for isolating and/or purifyng Aβ oligomer reactive antibodies.

Enrichment of Oligomer Reactive Antibodies

The present invention provides a method of enriching for oligomer reactive antibodies.

The present invention uses CAPS prepared by the method described above as ligands for enriching a biological sample for oligomer reactive antibodies. In one embodiment, a sample of oligomer reactive antibodies is enriched by affinity purification using isolated cross-linked oligomers. The present invention uses cross-linked oligomer affinity matrix of the present invention for enriching a sample for oligomer reactive antibodies.

Generally, a biological sample, such as a sample of commercially available IGIV or donor plasma, contains only a small amount (˜0.1%) of oligomer reactive antibodies. The inventors have found that a biogical sample may be enriched for oligomer reactive antibodies using an oligomer conjugated affinity column. For example, a sample of IGIV isolated from an oligomer affinity column is enriched for binding oligomers as compared to or relative to the starting material. The present invention provides oligomer reactive antibodies or fragments thereof enriched for oligomer binding. Such enrichment may comprise about a 10%, 20%, 50%, 75%, 100%, 200%, 400% or more increase in binding compared to the starting material. In another embodiment, such enrichment may comprise about a 2-fold, 3-fold, 4-fold, 5-fold, 7-fold, 10-fold, 20-fold, 50-fold, 100-fold, 500-fold or more binding compared to the starting material. In still another embodiment, the purified fraction may comprise about 1%, 5%, 10%, 25%, 50%, 75%, 80% or more oligomer reactive antibodies. IGIV enriched or concentrated for oligomer binding may be obtained by various affinity purification methods.

As an example, the present invention provides enriching AD oligomer reactive antibodies from IGIV by isolating Aβ oligomer reactive antibodies from IGIV using an oligomer affinity column. The isolated Aβ oligomer antibodies were enriched about 15 fold.

The present invention is based in part on the finding that using Aβ conformer affinity chromatography (using fibrils, CAPS, and monomers as the substrates), human sera was found to contain antibodies that are reactive against a limited number of common conformational epitopes on Aβ fibrils and CAPS, with negligible binding to the solution-phase monomeric peptide. The Aβ reactive antibodies eluted off CAPS and fibril columns appear to consist of diverse IgG populations since, each preparation binds preferentially against the Aβ conformer used for isolation, and in the presence of human plasma, CAPS isolated antibodies retained more activity against aggregated Aβ than fibril-purified IgGs. In other words, Aβ reactive antibodies eluted from an oligomer column has a higher affinity for oligomers, while Aβ reactive antibodies eluted from a fibril column has a higher affinity for fibrils.

Oligomer reactive antibodies recognize one or more conformational epitopes expressed on various oligomers and fibrils, such as LC fibrils, Aβ fibrils, CAPS, or wild-type and F19P. However, these antibodies did not bind these molecules in their native solution-phase states.

Uses of Compositions Comprising Oligomer Reactive Antibodies

The present invention is also based in part on results indicating that oligomer reactive antibodies cross-react with amyloidogenic oligomers by, presumably, binding to the same conformational epitope. The procedures and resultant reagents, described above and in the examples, can be used for diagnostic and therapeutic purposes for subjects with AD and other amyloid disorders-such as AIAPP and AL amyloidosis.

Recently, Lee et al. 2006 disclosed that passive immunization with a conformation-selective monoclonal antibody improved learning and memory in transgenic mice models of AD. Specifically, Lee et al. showed that transgenic mice treated with a novel monoclonal antibody (one that preferentially recognized a conformational epitope present in dimeric, small oligomeric, and higher order Aβ structures, but not the full length Aβ precursor protein or C-terminal AO protein fragments) displayed significantly improvements in spatial learning and memory relative to control mice. These results suggest that Aβ oligomers may be pathologic culprits for causing cognitive decline in AD.

The present invention provides compositions comprising oligomer reactive antibodies, fragments thereof, and compositions comprising antibodies or fragments thereof enriched for binding to amyloidogenic oligomers for treating diseases and conditions associated with amyloid deposition. The oligomer reactive antibodies and fragments thereof bind amyloidogenic oligomers. The oligomer reactive antibodies and fragments thereof prepared by the methods of the present invention may be used to neutralize the cytotoxic effect of oligomers in subjects in need thereof. Generally, oligomers are more cytotoxic than fibrils. Accordingly, the oligomer reactive antibodies play a role in clearing the soluble pool of oligomers and provide beneficial effect in patients suffering from amyloidosis.

In one embodiment of the invention, the present invention provides a method of treating a subject having amyloid deposition comprising administering to the subject a therapeutically effective amount of oligomer reactive antibodies or fragments thereof, wherein the oligomer reactive antibodies or fragments thereof bind amyloidogenic aggregates.

In another embodiment, the present invention provides a method of neutralizing the cytotoxic effects of amyloidgenic oligomers in a subject in need thereof comprising administering to the subject an effective amount of oligomer reactive antibodies or fragments thereof to bind oligomers, and allowing the antibodies or fragments thereof to bind amyloidogenic oligomers, thereby neutralizing or clearing the pool of soluble cytotoxic oligomers.

Moreover, the present invention provides a method of inhibiting the formation of amyloid deposits in a subject comprising administering to the subject an effective amount of oligomer reactive antibodies or fragments thereof to inhibit formation of amyloid deposits, and allowing the oligomer reactive antibodies or fragments thereof to bind amyloid-forming precursor protein, thereby inhibiting the formation of amyloid deposits. Oligomer reactive antibodies bind both oligomers and fibrils to inhibit amyloid growth by preventing fibril growth.

Further, the present invention provides a method of modulating the formation of amyloid deposits in a subject comprising administering to the subject an effective amount of oligomer reactive antibodies or fragments thereof to modulate formation of amyloid deposits, and allowing the antibodies or fragments thereof to bind the oligomer in the amyloid deposit, thereby modulating formation of amyloid deposits.

As an alternate embodiment, the present invention also provides compositions for diagnostic methods comprising oligomer reactive antibodies enriched for binding amyloidogenic oligomers. The present invention provides a method of detecting amyloid deposits in a subject comprising administering to the subject an effective amount of oligomer reactive antibodies or fragments thereof to detect amyloid deposits and allowing the oligomer reactive antibodies or fragments thereof to bind amyloidogenic oligomers, and detecting amyloid deposits.

The present invention also provides a method of imaging amyloid deposits in a subject comprising administering to the subject an effective amount of oligomer reactive antibodies or fragments thereof to image amyloid deposits and allowing the oligomer reactive antibodies or fragments thereof to bind amyloidogenic oligomers, and obtaining an image of the amyloid deposits.

Pharmaceutical Compositions of Antibodies

The present invention provides pharmaceutical composition or formulations comprising therapeutically effective amount of oligomer reactive antibodies, such as cross-linked Aβ oligomer reactive antibodies, for the treatment of amyloidosis in a subject or patient. The compositions could be used to inhibit, detect, image and modulate the formation of amyloid deposits in a subject. The antibody compositions of the present invention may be enriched for binding oligomers.

In one embodiment, the antibodies of the present invention are isolated from IGIV, blood, peritoneal fluid, or other biological fluids or samples that contain sufficient quantities of the antibodies.

The oligomer reactive antibodies of the present invention and fragments thereof are prepared by methods described above. The oligomer reactive antibodies or fragments thereof may be enriched for binding amyloidogenic oligomers.

The dosage of oligomer reactive antibodies and the method of administration will vary with the severity and nature of the particular condition being treated, the duration of treatment, the adjunct therapy used, the age and physical condition of the subject of treatment and like factors within the specific knowledge and expertise of the treating physician. However, single dosages for intravenous and intracavitary administration can typically range from 400 mg to 2 g per kilogram body weight, preferably 2 g/kg (unless otherwise indicated, the unit designated “mg/kg” or “g/kg”, as used herein, refers to milligrams or grams per kilogram of body weight). The preferred dosage regimen is 400 mg/kg/day for 5 consecutive days per month or 2 g/kg/day once a month. The oligomer reactive antibodies enriched for binding amyloidogenic oligomers of the present invention are effective for in vivo use.

In another embodiment of this invention, the amyloid reactive antibodies of the present invention are administered via the subcutaneous route. The typical dosage for subcutaneous administration can range from 4 mg to 20 mg per kg body weight.

According to the present invention, oligomer reactive antibodies may be administered as a pharmaceutical composition containing a pharmaceutically acceptable carrier. The carrier must be physiologically tolerable and must be compatible with the active ingredient. Suitable carriers include sterile water, saline, dextrose, glycerol and the like. In addition, the compositions may contain minor amounts of stabilizing or pH buffering agents and the like. The compositions are conventionally administered through parenteral routes, with intravenous, intracavitary or subcutaneous injection being preferred.

Detecting and Imaging Amyloid Deposits

The present invention further provides a method of detecting and imaging amyloid deposits using oligomer reactive antibodies prepared according to the methods of the present invention. The method of this invention determines the presence and location of amyloid deposits in an organ or body area, for example the brain, of a subject. The present method comprises administration of a detectable quantity or an imaging effective quantity of oligomer reactive antibodies, to a subject or patient. A “detectable quantity” means that the amount of the detectable compound that is administered is sufficient to enable detection of binding of the compound to amyloid. An “imaging effective quantity” means that the amount of the detectable compound that is administered is sufficient to enable imaging of binding of the compound to amyloid.

Oligomer reactive antibodies may be tagged with an diagnostic or imaging agent known in the art, such as radionuclides, enzymes, dyes, fluorescent dyes, gold particles, iron oxide particles and other contrast agents including paramagnetic molecules, x-ray attenuating compounds (for computed tomography (CT) and x-ray) contrast agents for ultrasound. Appropriate agents for imaging amyloid deposits include iron oxide particles, dyes, fluorescent dyes, nuclear magnetic resonance (NMR) labels, scintigraphic labels, gold particles, positron emission tomography (PET) labels, ultrasound contrast media, and CT contrast media. A variety of different types of substances can serve as the reporter group for tagging IGIV, including but not limited to enzymes, dyes, radioactive metal and non-metal isotopes, fluorogenic compounds, fluorescent compounds, etc.

Methods for preparation of antibody conjugates of the oligomer reactive antibodies (or fragments thereof) of the invention useful for detection, monitoring are described in U.S. Pat. Nos. 4,671,958; 4,741,900 and 4,867,973, the contents of which are hereby incorporated by reference. Also known in the art is the method of using monoclonal antibodies as probes for imaging of Aβ (WO 89/06242 and U.S. Pat. No. 5,231,000).

The invention employs tagged oligomer reactive antibodies which, in conjunction with non-invasive neuroimaging techniques such as magnetic resonance spectroscopy (MRS) or imaging (MRI), or gamma imaging such as PET or single-photon emission computed tomography (SPECT), or CT, x-ray, optical or infrared imaging, and ultrasound, are used to quantify amyloid deposition in vivo. The term “in vivo imaging” refers to any method which permits the detection of labeled antibodies, such as IGIV.

For purposes of in vivo imaging, the type of detection instrument available is a major factor in selecting a given label. For instance, radioactive isotopes such as ¹²⁵I are particularly suitable for in vivo imaging in the methods of the present invention. The type of instrument used will guide the selection of the radionuclide or stable isotope. For instance, the radionuclide chosen must have a type of decay detectable by a given type of instrument. Another consideration relates to the half-life of the radionuclide. The half-life should be long enough so that it is still detectable at the time of maximum uptake by the target, but short enough so that the host does not sustain deleterious radiation. The radiolabeled compounds of the invention can be detected using nuclear imaging wherein emitted radiation of the appropriate energy is detected. Methods of nuclear imaging include, but are not limited to, SPECT and PET. Preferably, for SPECT detection, the chosen radiolabel will lack a particulate emission, but will produce a large number of photons in a 140-200 keV range. For PET detection, the radiolabel will be a positron-emitting radionuclide such as ¹⁹F which will annihilate to form two 511 keV gamma rays which will be detected by the PET camera.

The methods of the present invention may use isotopes detectable by nuclear magnetic resonance spectroscopy for purposes of in vivo imaging and spectroscopy. Elements particularly useful in magnetic resonance spectroscopy include ¹⁹F, Gd and ¹³C.

Suitable radioisotopes for purposes of this invention include beta-emitters, gamma-emitters, positron-emitters, and x-ray emitters. These radioisotopes include ¹³¹I, ¹²³I, ^{99m}Tc, ¹¹¹In, ¹²⁴I, ¹⁸F, ¹¹C, ⁷⁵Br, and ⁷⁶Br. Suitable stable isotopes for use in Magnetic Resonance Imaging (MRI) or Spectroscopy (MRS), according to this invention, include ¹⁹F, Gd and ¹³C. Suitable radioisotopes for in vitro quantification of amyloid in homogenates of biopsy or post-mortem tissue include ¹²⁵I, ¹³¹I, ¹²³I, ^{99m}Tc, ¹⁴C, and ³H. The preferred radiolabels are ¹¹C, ¹²⁴I or ¹⁸F for use in PET in vivo imaging, ¹²³I, ^{99m}Tc, ¹¹¹In or ¹²⁵I for use in SPECT imaging, ¹⁹F or Gd for MRS/MRI, and ¹²⁵I, ³H or ¹⁴C for in vitro studies. However, any conventional method for visualizing diagnostic probes may be utilized in accordance with this invention.

The method may be used to diagnose AD or other diseases or conditions related to amyloidosis. This technique would also allow longitudinal studies of amyloid deposition in human populations at high risk for amyloid deposition such as Down's syndrome, familial AD, and homozygotes for the apolipoprotein E4 allele (Corder et al., 1993). A method that allows the temporal sequence of amyloid deposition to be followed can determine if deposition occurs long before dementia begins or if deposition is unrelated to dementia. This method can be used to monitor the effectiveness of therapies targeted at preventing amyloid deposition.

Generally, the dosage of the detectably labeled oligomer reactive antibodies will vary depending on considerations such as age, condition, sex, and extent of disease in the patient, contraindications, if any, concomitant therapies and other variables, to be adjusted by a physician skilled in the art.

Administration to the subject may be local or systemic and accomplished intravenously, intraarterially, intrathecally (e.g. via the spinal fluid) or the like. Administration may also be intradermal or intracavitary, depending upon the body site under examination. After a sufficient time has elapsed for the oligomer reactive antibodies to bind with the amyloid, for example 30 minutes to 48 hours, the area of the subject under investigation is examined by routine imaging techniques such as MRS/MRI, SPECT, planar scintillation imaging, PET, and any emerging imaging techniques, as well. The exact protocol will necessarily vary depending upon factors specific to the patient, as noted above, and depending upon the body site under examination, method of administration and type of label used; the determination of specific procedures would be routine to the skilled artisan.

Vaccines

The present invention provides vaccines for treating and preventing amyloidosis. The vaccine comprises an immunologically effective amount of oligomer reactive antibodies or fragments thereof and a pharmaceutically acceptable carrier. Moreover, the vaccine formulation of the present invention may also contain an adjuvant for stimulating the immune response and thereby enhancing the effect of the vaccine. The adjuvant may be selected from the group consisting of Freund's, BCG (bacilli Calmette-Guerin), Corynebacterium parvum, aluminum hydroxide (ALUM), lysolecithin, pluronic polyols, polyanions, and dinitrophenol.

The vaccine is administered to patients in need thereof. Vaccines of the present invention may be administered by any convenient method for the administration of vaccines including oral and parenteral (e.g. intravenous, subcutaneous or intramuscular) injection. The treatment may consist of a single dose of vaccine or a plurality of doses over a period of time. The vaccine of the present invention may include cross-linked oligomer reactive antibodies for passive immunization of the patient in need thereof. Alternatively, the vaccine of the present invention may include corss-linked oligomers for active immunization of a patient in need thereof.

As an example, the vaccine of the present composition comprises Aβ oligomer reactive antibodies or fragments thereof and a pharmaceutically acceptable carrier. The vaccine of the present invention may also comprise an adjuvant.

Kits for Preparing and Using Cross-linked Oligomers

The present invention provides kits for preparing and using cross-linked oligomers. In one embodiment, the kit comprises catalysts for cross-linking oligomers, such as but not limited to HRP and copper sulfate or copper chloride. HRP may be conjugated to a matrix or resin. The kit may contain blocking agents such as BSA or gelatin. The kit also may contain reagents for precipitating the cross-linked oligomer, such as but not limited to copper sulfate. The kit may also contain reagents for removing the catalyst or copper ions, such as but not limited to guanidine hydrochloride and EDTA. The kit may also include reagents for solubilizing the oligomers prior to cross linking, such as but not limited to TFA and HFIP.

In another embodiment, the kit contains cross-linked oligomers, such as cross-linked Aβ oligomers or other oligomers associated with amyloidosis, and means for isolating or purifyng oligomer reactive antibodies that bind to amyloidogenic oligomers or enriching a sample for such antibodies. The kit may include means and reagents for affinity purification of the oligomer reactive antibodies, such as an affinity matrix containing cross-linked oligomers conjugated to resin. Alternatively, the kit may include means and reagents for enriching oligomer reactive antibodies for binding amyloidogenic oligomers.

Kits for Using Oligomer Reactive Antibodies

The present invention also provides kits for treating, preventing, diagnosis, prognosis, monitoring, or detecting amyloidosis in a subject. The kit may contain antibodies isolated by the methods of the present invention. The antibodies may be isolated using cross-linked oligomers as the ligand via affinity purification.

The oligomer reactive antibodies in the kit can be tagged with a label. Alternatively, other components can be included in the kit for tagging the oligomer reactive antibodies. The present invention also contemplates kits comprising other components for treating subjects suffering from conditions or diseases related to amyloidosis, for preventing, diagnosing and monitoring the formation of amyloid deposits in a subject, and determining the prognosis of the subject. In one embodiment, the components of the kit are packaged either in aqueous medium or in a lyophilized form.

In a further embodiment, the kit may comprise a container with a label. Suitable containers include, for example, bottles, vials, and test tubes. The containers may be formed from a variety of materials such as glass or plastic. The container may comprise materials desirable from a commercial and user standpoint, including buffers, diluents, filters, needles, syringes, and package inserts with instructions for use.

The oligomer reactive antibodies in the kit may be packaged with a container for diagnosing or detecting amyloid deposits in a patient or for treating a patient. The kit may contain a label, such as a radioactive metal ion or a moiety for attaching to oligomer reactive antibodies. The label can be supplied either in fully conjugated form, in the form of intermediates or as separate moieties to be conjugated by the user of the kit.

Aβ Peptide

Amyloid beta (Aβ or Abeta) is a peptide of 39-43 amino acids that is the main constituent of amyloid plaques in the brains of AD patients. Similar plaques appear in some variants of Lewy body dementia and in inclusion body myositis, a muscle disease. AO also forms aggregates coating cerebral blood vessels in cerebral amyloid angiopathy. These plaques are composed of a tangle of regularly ordered fibrillar aggregates called amyloid fibers, a protein fold shared by other peptides such as prions associated with protein misfolding diseases.

Aβ is formed after sequential cleavage of the amyloid precursor protein (APP), a transmembrane glycoprotein of undetermined function. APP can be processed by α-, β-, and γ-secretases; Aβ protein is generated by successive action of the β and γ secretases. The γ secretase, which produces the C-terminal end of the Aβ peptide, cleaves within the transmembrane region of APP and can generate a number of isoforms of 39-43 amino acid residues in length. The most common isoforms are Aβ40 and Aβ42; the shorter form is typically produced by cleavage that occurs in the endoplasmic reticulum, while the longer form is produced by cleavage in the trans-Golgi network (Hartmann et al., 1997). The Aβ40 form is the more common of the two, but Aβ42 is the more fibrillogenic and is thus associated with disease states. Mutations in APP associated with early-onset AD have been noted to increase the relative production of Aβ42, and thus one suggested avenue of AD therapy involves modulating the activity of 0 and γ secretases to produce mainly Aβ40 (Yi et al. 2007).

The present invention provides a method of treating, preventing, monitoring, and diagnosing AD comprising administering Aβ oligomer reactive antibodies to patients in need thereof. The Aβ oligomer reactive antibodies are made by the methods described above. The Aβ oligomer reactive antibodies bind to the oligomers and neutralize the toxic effects of the oligomers in the patient. The antibodies can modulate and inhibit the formation of amyloid deposits in AD patients.

The oligomer reactive antibodies isolated by the methods of the present invention may be used to treat, prevent, monitor, and diagnose disorders associated with formation of aggregated proteins, for example, amyloidosis and neurodegenerative diseases.

Without further description, it is believed that one of ordinary skill in the art can, using the preceding description and the following illustrative examples, make and utilize the claimed invention. The following working examples therefore, specifically point out embodiments of the present invention, and are not to be construed as limiting in any way the remainder of the disclosure. Rather, they should be construed to encompass any and all variations which become evident as a result of the teaching provided herein.

### EXAMPLES

### Example 1

Materials and Methods

Reagents. >90% pure Aβ40 (amino acids 1-40 of SEQ ID NO: 2) and Aβ42 (NH₂-DAEFRHDSGY EVHHQKLVFF AEDVGSNKGA IIGLMVGGVVIA-COOH, SEQ ID NO: 2) were obtained from Quality Controlled Biochemicals (QCB; http://www.qcb.com/services/cps.htm). Trifluoroacetic acid (TFA) and ImmunoPure Horseradish peroxidase (HRP), H₂O₂ (30% in water), and 1,1,1,3,3,3-hexafluoro-2-propanol (HFIP) were from Pierce, Fisher, and ACROS Organics, respectively. High-binding plates were purchased from Corning Costar. Europium (Eu³⁺) conjugated streptavidin and enhancement solution were purchased from Perkin Elmer. Antibodies were obtained against the N-terminus (MAB1560, Chemicon Int.), middle portion (MAB1561, Chemicon), and the C-terminus (mAb(9F1), Calbiochem) of Aβ40. Biotinylated goat anti-human IgG (γ-specific) and mouse anti-HRP was from Sigma and Research Diagnostics Incorp., respectively. Amyloid fibril-reactive enriched IGIV was prepared using our standard V_{λ}6 JTO fibril affinity column (O'Nuallain et al., 2006). The blocking agents, essentially-fatty acid free bovine serum albumin, Starting Block, and Protein-Free Block were purchased from Sigma and Pierce, respectively. Gentle Ag/Ab Elution Buffer was purchased from Pierce. All other reagents were of analytical grade.

Preparation of Aβ stocks. Soluble Aβ peptide was prepared using sequential treatments with TFA and HFIP (O'Nuallain et al., 2006). Soluble Aβ40 was prepared by exposing ˜0.25 mg peptide per tube to sequential applications of TFA and HFIP and then organic solvents evaporated under an argon stream. Trace volatile solvents were then removed under high vacuum, and the peptide residue dissolved in 2 mM NaOH, followed immediately by addition of 10×PBS to 1×. To remove any residual aggregates, the sample was centrifuged (51,500×g, 17 h at 4° C.), and the supernatant was carefully removed and analyzed for Aβ40 concentration by reverse-phase HPLC (Agilent SB-C₃ column) from the peak area of the A₂₁₅ absorbance trace, using a Aβ standard curve from peptide that was calibrated by amino acid composition analysis (Kheterpal et al., 2001).

Soluble Aβ42 peptide was also prepared by sequential exposure to TFA and HFIP. ˜1 mg/ml Aβ42 in a glass vial was dried off, HFIP added to the same volume, and 75 μl of the sample (˜75 μg Aβ) added into polypropylene tubes. The samples were evaporated under argon, lyophilized for 1 h, and 1 ml 2 mM NaOH added. Samples were then pooled into 4 ml amounts, snap-frozen (liquid nitrogen), and lyophilized overnight. PBS (4 ml of 1×) was added to each sample, transferred to polycarbonate ultracentrifuge tubes, and centrifuged at 302,000×g for 1 h at 4° C. The peptide concentration of the pooled supernatants (−0.035 mg/ml) was determined by reverse-phase HPLC.

Soluble Aβ was also prepared by dissolving the peptide in 2 mM NaOH, and any aggregated peptide removed by centrifugation. After 5 min, 10×PBS was added to 1× and the sample sonicated using a probe sonic disruptor (Teledyne/Tekmar) for 3 min on ice followed by centrifugation at 20,800×g for 30 min at 4° C. Peptide concentration in the supernatant was then determined by reverse-phase HPLC.

Unless, indicated otherwise, soluble Aβ that was used in our experiments was prepared by the TFA/HFIP protocol.

Preparation of cross-linked redox-modified Aβ oligomers: Two reagents, copper and HRP, known to catalyze dityrosine cross-linked Aβ oligomer formation, were used to generate CAPS (Galeazzi et al., 1999; Moir et al., 2005; Atwood et al., 2004; Yoburn et al., 2003; Ali et al., 2006). Reaction products were analyzed and quantified by SDS PAGE using 4-12% Bis Tris precast gels (Invitrogen Corp.) and MES running buffer. Gels were stained with Silver Snap (Pierce) or Coomassie R-250 stain (Pierce) and imaged using a Chemi-imager 4000 low light imaging system (Alpha Innotech Corp.).

Copper induced oligomers: CAPS were prepared from the soluble peptide by incubating, with or without agitation (microspin bar), soluble Aβ (˜0.2 mg/ml) with 0-100 mM CuSO₄ or CuCl₂ and 250 μM-1 mM H₂O₂ in PBS at 37° C. for 1-72 h. Alternatively, in an attempt to increase the efficiency of dityrosine cross-linking, sonicated Aβ fibrils were used as the substrate in an agitated reaction (Yoburn et al., 2003). Reaction products were analyzed by SDS PAGE by dissolving the insoluble product in neat TFA for 10 min, blown dry with argon, and solubilized by addition of 10 μl of 2 mM NaOH and 2×PBS added to 1×.

HRP induced oligomers: CAPS were prepared from soluble Aβ (0.03-0.2 mg/ml) incubated with 0-9 μM HRP and 250-650 μM H₂O₂ in PBS at 37° C. for 1-72 h. Alternatively, soluble Aβ was incubated with HRP conjugated to NHS-activated Sepharose 4 fast flow beads (GE Healthcare). Bead conjugation was carried out using 5 mg of HRP per ml of bead volume, as per manufacturer's instructions (GE Healthcare). HRP-conjugated beads were used directly or preblocked with blocking agent, 1% BSA, 1% gelatin, Starting Block, or Protein Free Block.

Purification of HRP induced cross-linked Aβ oligomers. Size exclusion gel fractionation (SEC). A typical SEC experiment involved loading 400 μl of 0.2 fig/ml CAPS reaction sample onto a 10 ml column (Superdex™ 75 or Sephacryl S200 (GE Healthcare)) that was pre-equilibrated with PBS. 1-ml fractions were collected, and the yield of peptide determined using a bicinchoninic acid colorimetric assay (micro-BCA, Pierce, Rockford, Ill.) with a BSA standard curve.

Reverse-phase HPLC. CAPS (60 μl of 0.1 mg/ml) were mixed with the same volume of 1% TFA and 100 μl was injected onto a Zorbax SB-C₃ column (Agilent Technologies) connected to a guard column (Agilent Technologies). The Aβ peptide was eluted and the yield determined as described above.

Copper precipitation of Aβ oligomers: The ability of copper to readily precipitate Aβ (Atwood et al., 2004) was used as a means to purify CAPS from HRP. Briefly, 1 mM CuSO₄ was added to the Aβ oligomer reaction product, and after gently mixing, the sample was immunediately aliquoted (1 ml per eppendorf tube), incubated for 2 h at room temperature and centrifuged at 20,800×g for 30 min at 4° C. The supernatant (mainly containing HRP) was removed and the Aβ pellet washed ×3 with PBS. Each wash cycle involved additions of 1 ml PBS to the pellet, dispersion by gentle pipetting and/or vortexing, and isolating the aggregated peptide by centrifugation. After washing, to ensure removal of residual HRP still bound to the Aβ precipitate, samples were mixed gently and incubated for 30 min with reagents or conditions known to disrupt protein-protein interactions (e.g., guanidine-HCl, urea, and high pH). The A, aggregates were resolubilzed by addition of 1 ml of 5 mM EDTA in 1×PBS for 2 h at room temperature followed by centrifugation as above. The preparations were dialyzed, using a 5000 MW cut-off membrane (Fisher), and used immediately or snap frozen (liquid N₂) and stored at ˜80° C. for up to 1 mo.

Biophysical analysis of purified cross-linked Aβ oligomers. Electrosprny ionization mass spectrometry. Purified CAPS (−0.2 mg/ml) were loaded onto a 20 μl loop on an Applied Biosystems (Foster City, Calif.) 173 Capillary HPLC and chromatographed using a reverse phase Aquapore 300 C₈ (150×0.5 mm) column with a gradient from 15% to 70% acetonitrile modified with 0.02% TFA. The gradient was developed over a period of 90 min with the eluent directed into the ion-spray of a PE-Sciex (Applied Biosystems) type 150 EX single quadrupole mass spectrometer. Masses were then determined using the Biomultiview software provided by the manufacturer.

Dityrosine and Thioflavin T fluorescence. Dityrosine fluorescence emission wavelength scans were determined using ˜0.2 mg/ml purified CAPS or monomer control in PBS with excitation at 320 nm and emission measured between 350-550 nm, using a Aminco Bowman series 2 spectrofluorimeter. Each thioflavin T (ThT) fluorescent measurement was carried out by diluting an aliquot of the reaction sample (equivalent to 5 μg Aβ) into a microtiter well that contained PBS and 30 μM ThT. ThT fluorescence was then monitored by excitation at 450 nm and emission at 482 nm using a FL600 fluorescence plate reader (Bio-Tex Instruments).

Electron micrographs: Electron micrographs (EM) of Aβ fibrils and CAPS samples were obtained using the University of Tennessee's EM facility. Specimens (˜0.2 mg/ml) were adsorbed onto carbon and formvar-coated copper grids and then negatively stained with 0.5% uranyl acetate. Stained samples were examined and photographed using a Hitachi H-800 instrument.

EuLISA. The dissociation-enhanced lanthanide fluoroimmunoassay incorporating europium (Eu³⁺)-streptavidin and time-resolved fluorometry (EuLISA) (O'Nuallain et al., 2006) was used to test the reactivity of anti-fibril enriched IGIV, unfractionated IGIV, or commercial anti-Aβ antibodies against Aβ monomer, CAPS, or fibrils coated (400-500 ng) on activated high-binding microtiter plate wells. After blocking with 1% BSA in PBS for 1 h at 37° C., wells were washed ×2 with PBS containing 0.05% sodium azide, followed by addition of biotinylated goat anti-human or anti-mouse IgG, and the plate incubated as before. After incubating at for 1 h at 37° C., the plate was washed and Eu³⁺-streptavidin conjugate added, followed by the releasing-enhancement solution. Antibody binding was detected by Eu³⁺ time-resolved fluorescence using a PerkinElmer Victor2 1420 Multilabel Counter. The amount (fM) of lanthanide released was calculated from a standard curve using known concentrations of Eu³⁺.

Western blot: Standard Western blot procedures were used to quantify and identify Aβ-containing bands as well as HRP present in CAPS reaction samples. Briefly, protein bands from oligomer samples run on 4-12% Bis Tris gel were transferred onto a PDVF membrane (Invitrogen Corp.) and after gentle shaking, the membrane was blocked with 1% BSA in PBS for 1 h at room temperature. Commercial mouse anti-Aβ (mixture of mAbs against N-terminal, C-terminal and the mid portion of Aβ) or anti-HRP antibody in blocking buffer containing 0.05% Tween 20 (assay buffer) was then added and the sample shaken. After 3 washes with PBS containing 0.05% Tween 20, the membrane was incubated with biotinylated goat anti-mouse IgG, washed again and alkaline phosphatase conjugated streptavidin added. The membrane was washed and the stain developed with Western Blue substrate (Promega). All blots were imaged using a Chemi-imager 4000 low light imaging system (Alpha Innotech Corp.).

Results

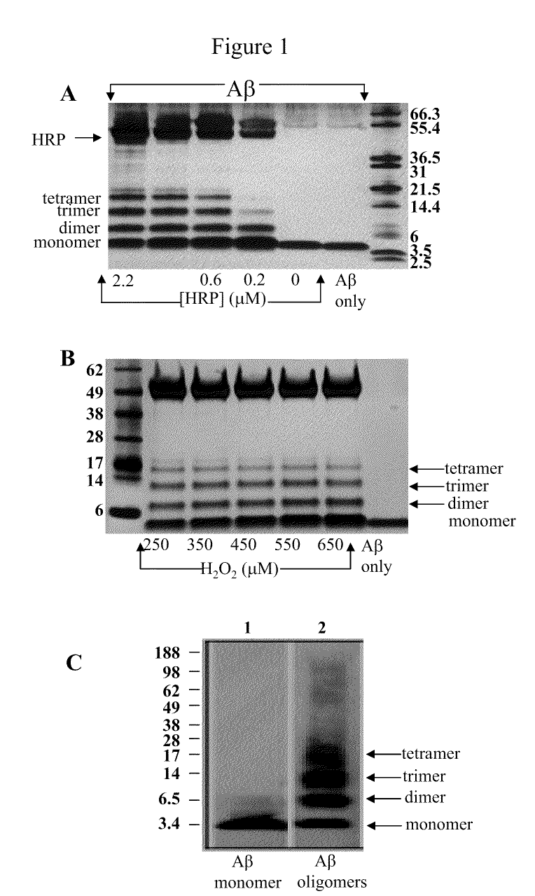

More efficient cross-linked Aβ40 oligomer formation was obtained with HRP than with copper. A discrete ladder of soluble SDS stable dimer, trimer and tetramer CAPS were observed when the redox modified peptide was obtained using 0.6-2.2 μM HRP (FIG. 1). The reaction was complete within 1 day and no change in aggregate yield (˜65%) occurred when the reaction was carried out for 1 to 3 days or with >1.1 μM HRP (FIG. 1A). Further experiments showed that this reaction was essentially complete within a few hours (data not shown). Additional attempts to increase the oligomer yield using higher concentrations of H₂O₂ (250-650 μM), or by dosing the reaction with fresh H₂O₂ proved unsuccessful (FIG. 1B). These results suggested that there is a proportion of Aβ40 that always remained unreactive (−20% by silver stain or ˜5-10% by Coomassie stain). The size distribution of SDS-stable oligomers that were obtained was very similar to that found by (Moir et al., 2005) from Western blot analysis of HRP-catalyzed Aβ oligomer formation under similar conditions (FIG. 1C).

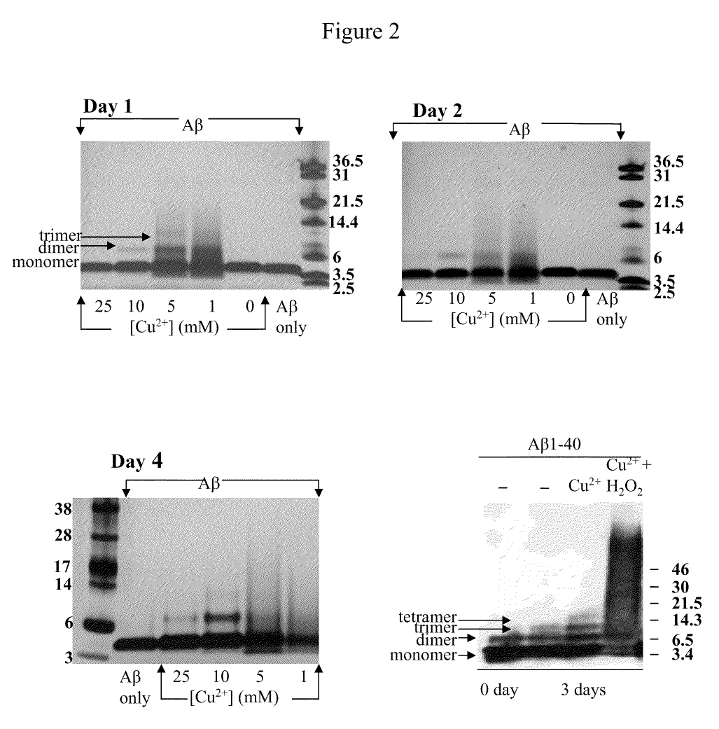

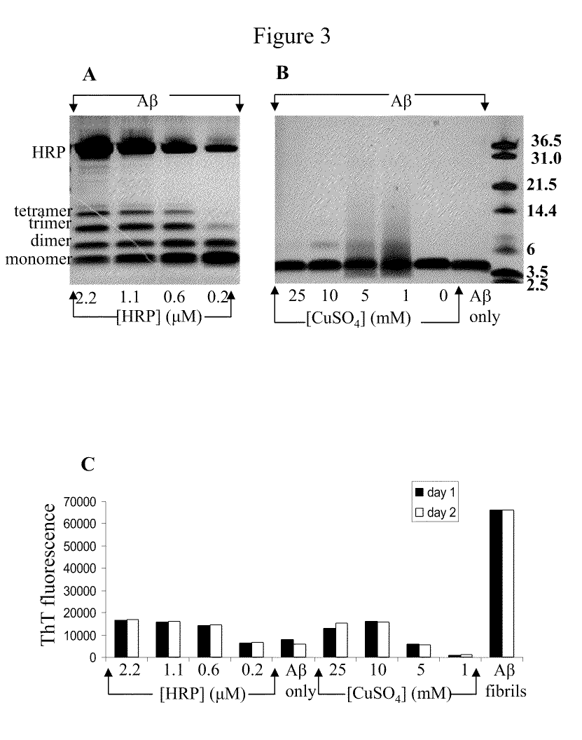

In contrast to results with HRP, only a small amount of SDS-stable CAPS (primarily insoluble dimer and trimer) were observed when CuSO₄ or CuCl₂ was the catalyst (FIGS. 2 & 3). In an attempt to increase the efficiency of aggregate production, Cu²⁺ catalyzed reactions were repeated with longer incubation times and higher Cu²⁺ concentrations. FIG. 2 shows that the reaction was complete within ˜1 day (incubating the reaction for up to 4 days had little effect on the product [<50% yield] and increasing the copper concentration [>5 mM] inhibited the reaction). Presumably, the ability of copper to readily precipitate AO hinders the metal's redox capacity. The low yield of Aβ aggregates with copper catalysis was somewhat unexpected given that (Atwood et al., 2004) have reported SDS-stable high molecular weight (dimers, trimers, tetramers, pentamers, etc.) Aβ oligomers by Western blot analysis under similar reaction conditions (FIG. 2). However, it is quite possible that the high molecular weight (>dimers) aggregates they detected were at a low concentration and would not have been evident by SDS PAGE analysis. Although, only small amounts of SDS-stable Aβ oligomers were obtained with Cu²⁺, the ThT fluorescence of these products was similar to that obtained with products formed using HRP (and ˜4 fold lower than the ThT fluorescence observed for the same weight of Aβ fibrils) (FIG. 3C). Because these products gave significant ThT fluorescence it is likely that these oligomers contain amyloid fibril-like higher ordered structure.

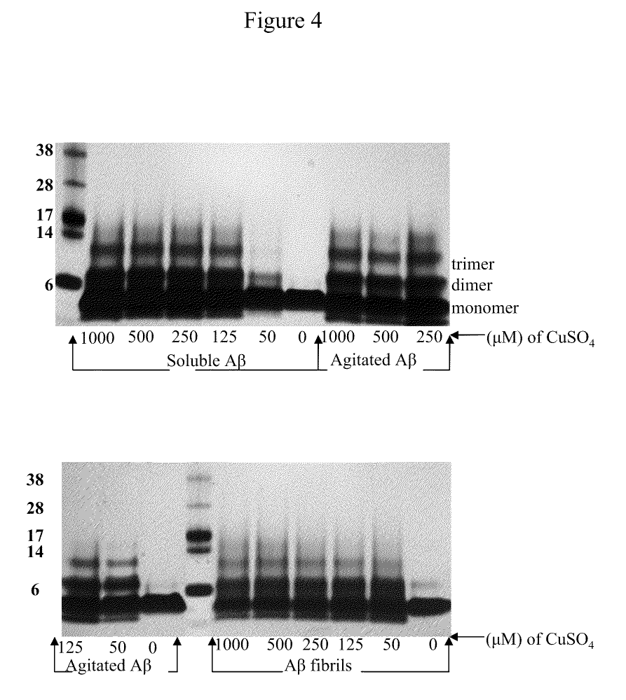

Copper was a more efficient catalyst when the Aβ40 substrate was less disaggregated: Because Yoburn et al. (2003) have shown that CAPS formation is more effective when the substrate is fibrillar, it was determined whether copper catalysis would also be more efficient with a more aggregated peptide substrate. Comparison of FIGS. 2 and 4 shows that a significantly higher yield (˜60%) of insoluble SDS-stable oligomers were obtained when the substrate was not thoroughly disaggregated by organic solvents (TFA HFIP, O'Nuallain et al., 2006). Instead, the peptide substrate was solubilized using 2 mM NaOH and any large insoluble aggregates removed before use by centrifugation. However, the yield of aggregates was not improved by agitating the reaction or by using Aβ fibrils as the substrate (FIG. 4).

HRP is the preferred catalyst for cross-linked Aβ40 oligomer formation: Taken together, results indicate that HRP is the preferred catalyst, because it gives the greatest reaction yield with a discrete ladder of soluble SDS stable dimer, trimer and tetramer Aβ oligomers; furthermore, the CAPS product is soluble, in contrast to the insoluble aggregate product formed by copper. Thus, further studies on the optimization and preparation of purified redox-modified Aβ aggregates utilized HRP.

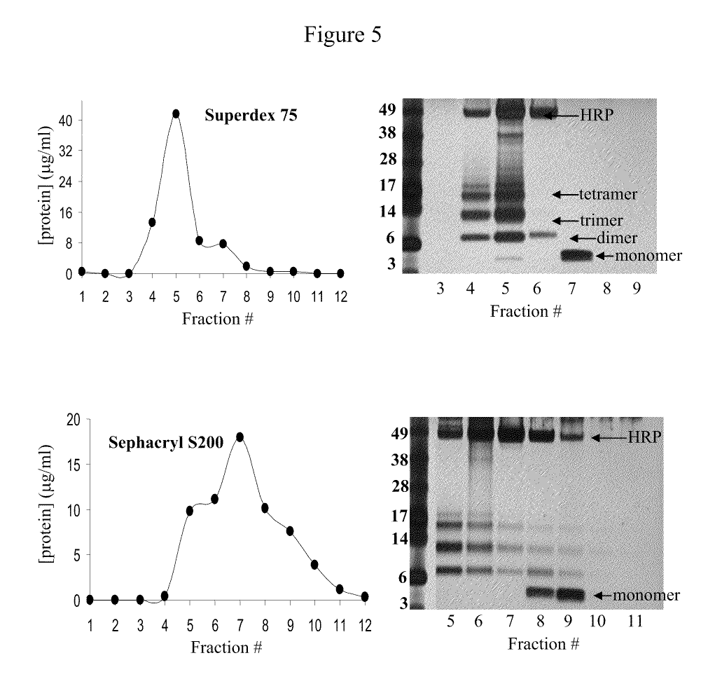

Determination of an optimal method for purifying cross-linked Aβ40 oligomers: Four methods (size exclusion chromatography (SEC), reverse-phase HPLC, HRP conjugated beads, and Aβ oligomer precipitation) were investigated as a means to obtain optimal purification. FIG. 5 shows that HRP co-eluted with CAPS when reaction samples were run on Superdex 75 and Sephacryl S200 SEC columns. The best separation was achieved using Sephacryl S200, suggesting that under native conditions these oligomers are high molecular weight species. Most importantly, SEC resulted in only a moderate oligomer yield (˜60%).

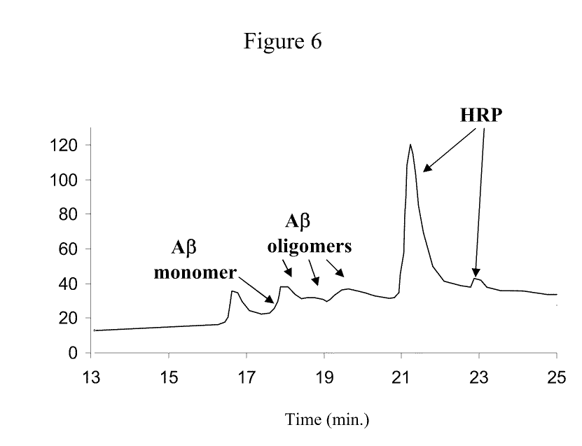

In contrast, reverse-phase HPLC proved to be a better method for purifying CAPS (FIG. 6). However, the yield was again moderate (˜70%) and this was attributed to oligomers sticking to the guard column. As shown with the chromatograph depicted in FIG. 6, a significant amount (˜10%) of Aβ in the reaction product eluted just before the monomeric peptide.

Conceivably, this fraction could have contained oxidized Aβ, Met35, and/or to the bound peptide that has less hydrophobic side chains exposed for column binding, due to higher-ordered peptide structure.

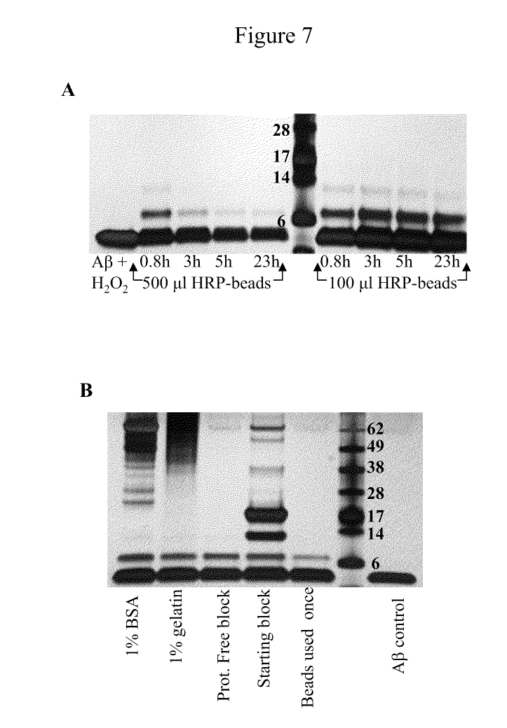

Comparison of FIGS. 1 and 7 shows that significantly less CAPS formation was obtained using HRP-conjugated beads than when HRP alone was the catalyst. The product mimicked the results obtained with copper. Only small amounts of SDS-stable Aβ dimers were formed with no increase in aggregate formation observed after a 3 h incubation period (FIG. 7). Control experiments with HRP-conjugated beads and free HRP, and 2,2′-azino-bis(3-ethylbenzthiazoline-6-sulphonic acid) as the substrate confirmed that the low efficiency with HRP-beads was not due to a loss of enzyme activity on bead conjugation.

Surprisingly, less soluble product was obtained when the reaction was repeated with a 5-fold greater amount of HRP-beads (FIG. 7A). This and the observation that less dense SDS PAGE Aβ gel bands were obtained with reactions carried out with higher amounts of HRP-beads suggests that the lack of aggregation was because Aβ oligomers stuck to the HRP-beads. Therefore, different blocking agents, such as BSA and gelatin were used to preblock beads before use. FIG. 7 shows that the supernatant from several oligomer reactions carried out with blocked HRP-beads contained high molecular weight protein bands in addition to an Aβ dimer band that was observed with the unblocked HRP-bead reaction. Of particular interest was the banding pattern obtained using HRP-beads pre-blocked with Starting Block (a protein based proprietary block; Pierce) (FIG. 7B).

However, control experiments with blocking agents alone confirmed that these additional bands were attributed to release of blocking agents from HRP-beads into the solution during sample incubation.

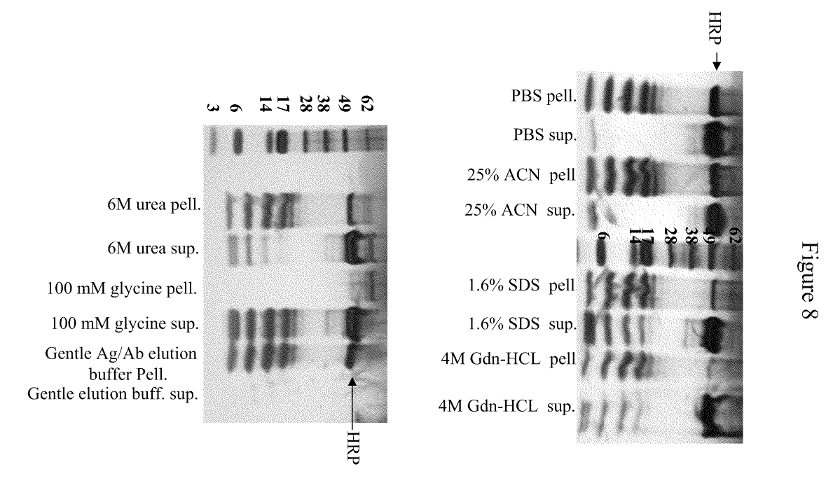

The final purification method investigated was based on copper's ability to selectively precipitate Aβ. FIG. 8 shows that Cu²⁺ precipitation of CAPS resulted in the removal of ˜80% HRP and oligomer yield was the highest of any purification method (>90%). Control experiments with HRP and CuSO₄ alone or combined together showed that HRP was not precipitated by copper. Instead, it was posited that the co-precipitation of Aβ and HRP results from the catalyst forming complexes with the peptide. Therefore, several agents and conditions known to block protein-protein interactions, such as guanidine-HCl, SDS, urea, high salt, and extreme pH were tested to identify an optimal agent for disrupting Aβ-HRP interactions. SDS PAGE analysis showed that the best candidates were guanidine-HCl (4 M) and urea (6M), although ˜20% and ˜10% of the Aβ pellet was dissolved with these treatments (FIG. 8). Notably, the Aβ/HRP precipitate was resolubilized when exposed to high pH buffer (100 mM glycine, pH 10.5) (FIG. 8). This could be due to a disruption of electrostatic forces that are important for copper-Aβ interactions.

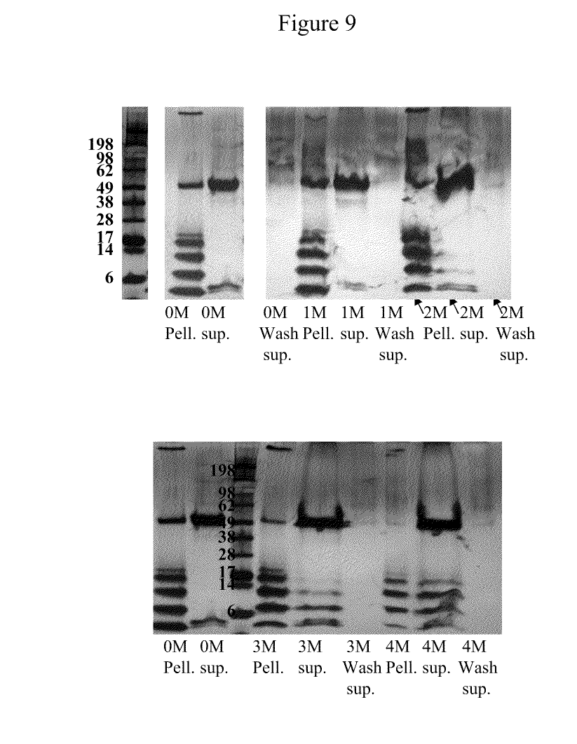

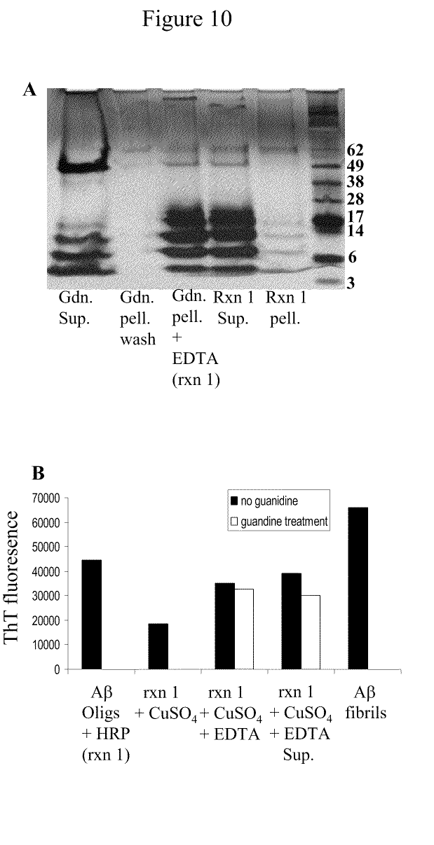

Although guanidine-HCl and urea were similarly effective at removing HRP from precipitated CAPS, the former was the preferred reagent for further study because it is a relatively inert molecule, unlike urea that breaks down into cyanate ions that can react with free amino groups (Harding et al., 1989). SDS PAGE analysis of the effects of 0-4 M guandine-HCl on the copper precipitated reaction product showed that 3 M guandine-HCl was the optimal concentration for removing peptide-bound HRP without significantly dissolving the peptide pellet (FIG. 9). Additional washes with PBS did not result in further solubilization of the pellet but did remove residual guanidine-HCl. The Aβ oligomer pellet was resolubilized with 5 mM EDTA in PBS (˜0.3 mg/ml) and after centrifugation, the supernatant containing purified (˜90%, the impurity being monomeric Aβ as evident by SDS PAGE) Aβ40 oligomers was obtained at a high yield (>70%). Any oligomer pellet could be readily resolubilized by the addition of a high pH buffer (200 mM glycine, PBS and 5 mM EDTA, pH 10.5). SDS PAGE and ThT fluorescence studies showed that purified Aβ40 oligomers had the same properties as the impure aggregates (FIG. 10). This finding suggested that purification per se does not alter oligomer morphology. The schematic in FIG. 13 summarizes the optimal protocol for oligomer purification.

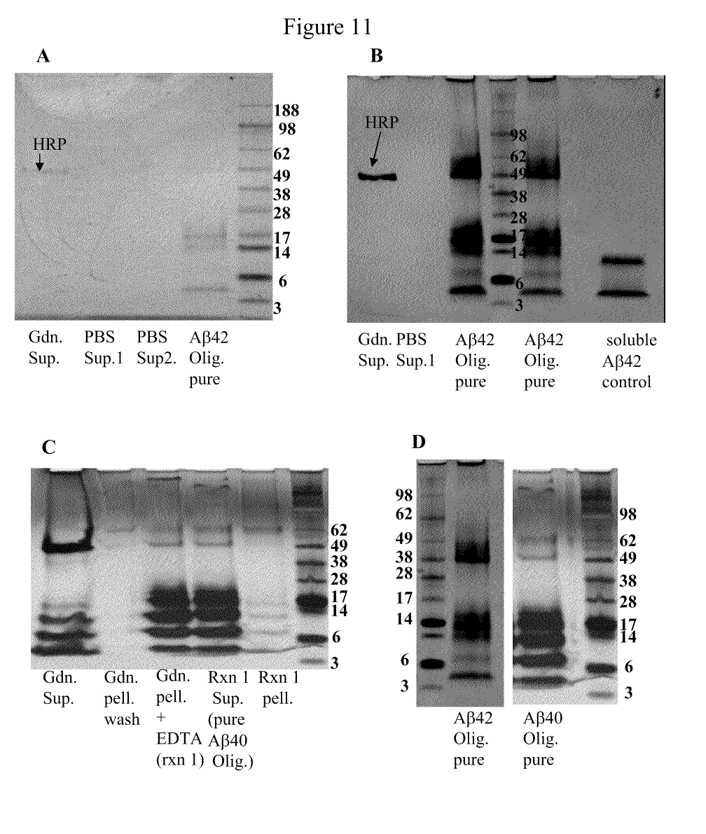

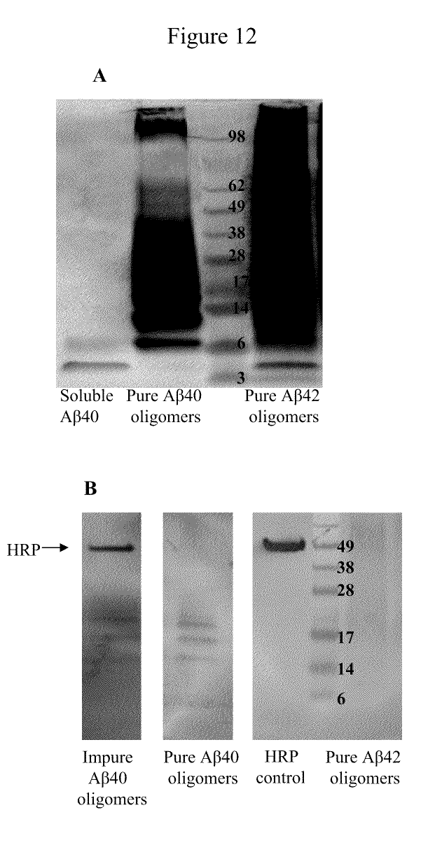

Aβ42 forms more higher molecular weight SDS-stable cross-linked oligomer species than Aβ40. SDS PAGE analysis of guanidine-HCl and PBS supernatants of washed Aβ42 oligomer pellets showed that, as with Aβ40 oligomers, HRP was removed from the pellet. However, the CAPS aggregates were more stable to guanidine-HCl as no low molecular weight species (Aβ42) appear to be present in the denaturant supernatant (FIG. 11). Furthermore, Aβ42 formed a greater proportion of higher molecular weight species than Aβ40 (FIG. 11). Western blot analysis with anti-Aβ and anti-HRP antibodies indicates that the highest molecular weight species (˜45 kDa) in purified Aβ40 and Aβ42 oligomer samples is Aβ and not HRP (FIG. 12).

Biophysical characterization of cross-linked Aβ oligomers: Initial biophysical characterization of CAPS by electrospray ionization mass spectrometry, dityrosine fluorescence, electron microscopy, thioflavin T fluorescence, Western blot analysis, and binding to anti-Aβ antibodies (enriched anti-fibril IGIV and commercial antibodies), suggested that these aggregates consisted of globular and protofibril-like assemblies that typify fibril assembly intermediates (Watson et al., 2005; Goldsbury et al., 2005; Walsh et al., 1999; Walsh et al., 1997).

Electrospray ionization mass spectral analysis confirmed that CAPS contained covalently cross-linked Aβ dimers and hexamers (FIG. 14). Presumably, these are cross-linked through dityrosines (Galeazzi et al., 1999; Atwood et al., 2004; Ali et al., 2006), as purified Aβ oligomers gave typical dityrosine fluorescence emission wavelength spectra with an emission maximum at ˜418 n by excitating at 320 nm (FIG. 15). In contrast, monomeric Aβ controls did not fluoresce at these wave-lengths (FIG. 15). Mass spectral analyses also confirmed that cross-linked Aβ molecules were of a molecular weight consistent with the unmodified peptide, covalently bound by dityrosine; further, no gross redox modification of the aggregated peptide was evidenced. However, due to low ionization of the peptide and the heterogenous nature of the oligomeric sample, it was not possible to determine if all Aβ oligomers (including trimers, and tetramers that were observed by SDS PAGE) were covalently cross-linked and unmodified.

Electron micrographs of purified CAPS showed that these molecules were globular and consisted of protofibril-like aggregates that were much larger than that observed by SDS PAGE and typified Aβ fibril assembly intermediates (Watson et al., 2005; Goldsbury et al, 2005; Walsh et al., 1999; Walsh et al., 1997) (FIG. 16). Additionally, Western blot analyses using a mixture of 3 commercial antibodies that each recognize an epitope in the N-terminal, C-terminal or mid portion of the Aβ peptide showed that oligomer preparations contained SDS-stable high molecular weight oligomers (>tetramers) that, presumably, were not at a high enough concentration to be detected by SDS PAGE (FIG. 17).

To determine whether purified CAPS contain amyloid fibril-like epitopes, EuLISA antibody binding curves were constructed using enriched anti-fibril IGIV against, Aβ fibrils, oligomers, and monomer. FIG. 18 shows that anti-fibril enriched IGIV has similar affinity for Aβ oligomers and fibrils (EC₅₀ values of ˜30 nM), but notably weaker binding to Aβ monomer (Ec₅₀ values of ˜1 μM). Taken together, these results were indicative of fibril-associated epitope(s) on purified cross-linked Aβ oligomers.

### Example 2

### Materials and Methods

Reagents. >90% pure Aβ40 (amino acids 1-40 of SEQ ID NO: 2) and Aβ42 (NH₂-DAEFRHDSGY EVHHQKLVFF AEDVGSNKGA IIGLMVGGVV IA-COOH; SEQ ID NO: 2) were obtained from Quality Controlled Biochemicals (QCB; http://www.qcb.com/services/cps.htm). Trifluoroacetic acid (TFA) and ImmunoPure Horseradish peroxidase (HRP), H₂O₂ (30% in water), and 1,1,1,3,3,3-hexafluoro-2-propanol (HFIP) were from Pierce, Fisher, and ACROS Organics, respectively. High-binding plates were purchased from Corning Costar. Europium (Eu³⁺) conjugated streptavidin and enhancement solution were purchased from Perkin Elmer. Biotinylated goat anti-human IgG (γ-specific) and mouse anti-HRP was from Sigma and Research Diagnostics Incorp., respectively. The IGIV preparation (Gammagard Liquid®) was from Baxter AG/Biosciences (Vienna, Austria). Amyloid fibril-reactive enriched IGIV was prepared using our standard V_{λ}6 JTO fibril affinity column (O'Nuallain et al. (2006)). The blocking agent, essentially-fatty acid free bovine serum albumin was purchased from Sigma. All other reagents were of analytical grade.

Aβ conformer preparation: Soluble Aβ and CAPS were prepared as described in Example 1. Aβ fibrils were generated as described previously (O'Nuallain et al. 2002). Briefly, the soluble, disaggregated (TFA/HFIP pretreated) Aβ peptide was dissolved in PBSA (0.25 mg/ml) and incubated at 37° C. with a seed consisting of 0.1% (by weight) sonicated Aβ fibrils. Based on thioflavin T fluorescence intensity, maximum fibril formation occurred within 5 to 7 days (Naiki et al. 1989; Levine et al. 1993). Fibrils were harvested by centrifugation, washed ×2 with PBSA, sonicated (2×30 sec bursts) with a probe sonic disrupter (Teledyne/Tekmar, Mason, Ohio), aliquoted, and stored at −20° C.

Preparation of Aβ40 conformer affinity columns: Each Aβ40 conformer (Fibrils, CAPS, or monomer) was linked covalently to an N-hydroxysuccinimide (NHS)-activated Sepharose® 4 fast-flow pre-activated agarose matrix with a mean bead size of 90 μm (Amersham Biosciences Corp., Piscataway, N.J.). For this procedure, a 10-ml packed bed volume of matrix (supplied as a suspension in 100% isopropanol) was washed ×3 with an equal amount of cold 1 mM HCl and centrifuged at 1000×g for 4 min at 4° C. Ten-ml of Aβ conformer (fibrils were sonicated with a probe sonic disrupter (Teledyne/Tekmar) before use) in PBS (˜1 mg/ml) was added to the medium and the mixture stirred gently at room temperature every 30 min. The coupling reaction was terminated 3 hrs later by addition of 0.1 M Tris-HCl, pH 7.5, to the centrifuged medium and, after another 3 hrs, the matrix was washed ×5, with each cycle consisting of 3 column volumes of 0.1 M Tris-HCl, pH 8.2, and one of 0.1M sodium acetate, pH 3.5. The final product was poured into a plastic polypropylene column (Pierce), washed ×4 with 10 ml of PBS, and stored at room temperature.

Preparation of Aβ40 conformer affinity-purified IGIV: IGIV (Gammagard Liquid®) was filtered to render the preparation aggregate-free, diluted with PBS to yield a final concentration of 10-20 mg ml, and loaded onto the appropriate Aβ40 conformer conjugated, PBS pre-equilibrated column. To remove any weakly binding or unbound (residual) protein, the column was washed with 40 ml of PBS and then the fibril-bound antibodies eluted in 1-ml portions using 0.1 M glycine buffer, pH 2.7; the fractions were neutralized by addition of 1 M Tris HCl, pH 9. The concentration of IgG in the Aβ conformer affinity-purified eluates and residual filtrates was determined based on absorbance at 280 nm, using an E₂₈₀^{1%} of 1.30 and a mol wt of 150000 daltons. Samples containing the enriched antibodies were pooled and concentrated with a PL-30 Centricon® (Millipore) apparatus and stored at 4° C. for up to 2 wks or maintained frozen at −20° C.

Europium-linked immunosorbant assay (EuLISA): As described in Appendix A, a dissociation-enhanced lanthanide fluoroimmunoassay (Diamandis et al. 1988) utilizing europium (Eu³⁺)-streptavidin and time-resolved fluorometry (DELFIA® system, Perkin Elmer Life Sciences, Boston, Mass.) was used to characterize the Aβ conformer reactivity of Aβ oligomer-isolated antibodies (O'Nuallain et al. 2002, O'Nuallain et al. 2006). All measurements in this and other assays were done in triplicate (error bars in the figures represent SD).

Antibody Production and Characterization: BALB/c mice were immunized ×5 with 50-μg injections of purified CAPS, generated from the Aβ40 peptide (see Appendix A), over a 3 mo. period. The reactivity of sera obtained 1 wk after the final injection against microtiter plate-immobilized Aβ40 oligomers was determined using our EuLISA, with both biotinyl-goat anti-mouse IgG and anti-IgM for detection.

Results

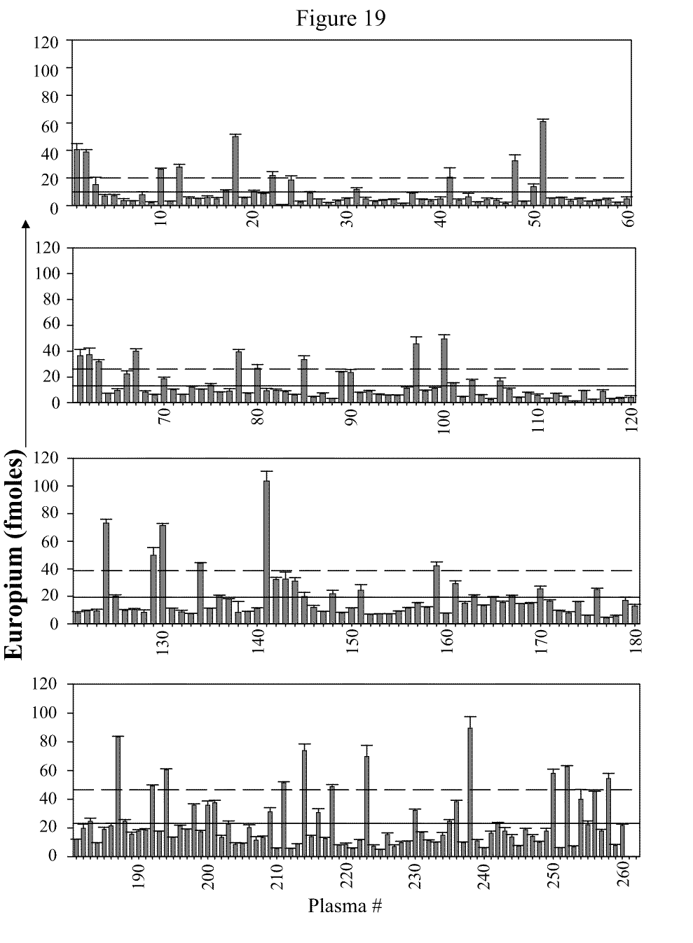

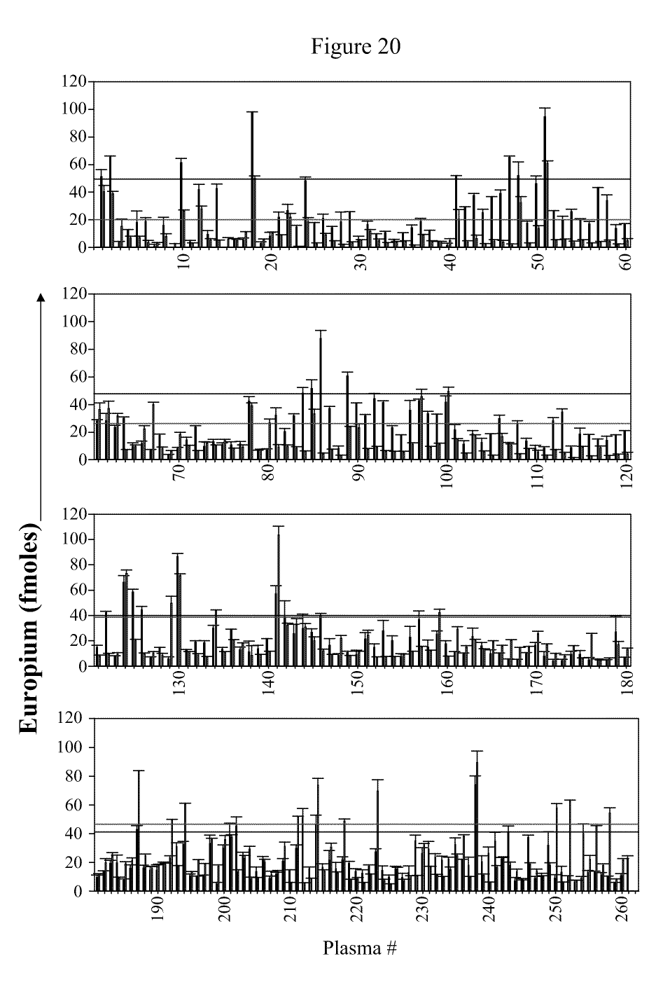

Human plasma and IGIV preparations contain antibodies that bind strongly to Aβ conformers, but weakly with Aβ monomer: Plasma from normal humans and an IGIV preparation was found to contain antibodies against purified CAPS (FIG. 19). Further plasma screening showed that there was a similar sera antibody response against Aβ40 fibrils and CAPS, but ˜20-fold lower signal was obtained against Aβ40 monomer (FIGS. 20 & 21) (Table 2).

Table 2 shows statistical comparison of EuLISA signals obtained for anti-LC fibril and anti-Aβ conformer reactivity in 262 (normal) human plasma samples

TABLE 2

| | LC (Jto) | Aβ40 | Aβ40 Oligomers | Aβ40 |
|---|---|---|---|---|
| | fibrils | fibrils | (CAPS) | monomer |
| Mean | 12.44 ± 11.5 | 22.19 ± 16.6 | 16.95 ± 16.7 | 2.52 ± 3.3 |
| Median | 9.98 | 17.62 | 10.82 | 1.5 |
| Min | 0 | 0 | 0.77 | 0 |
| Max | 103 | 98 | 103.5 | 23.2 |

These results indicate that naturally occurring human antibodies are reactive against common conformational epitope(s) on fibrils and CAPS, and that there are no specific (or at least at a high concentration) antibodies against the Aβ sequence. To further characterize naturally occurring Aβ oligomer-reactive antibodies, we isolated the reactive species in IGIV were isolated by affinity chromatography.

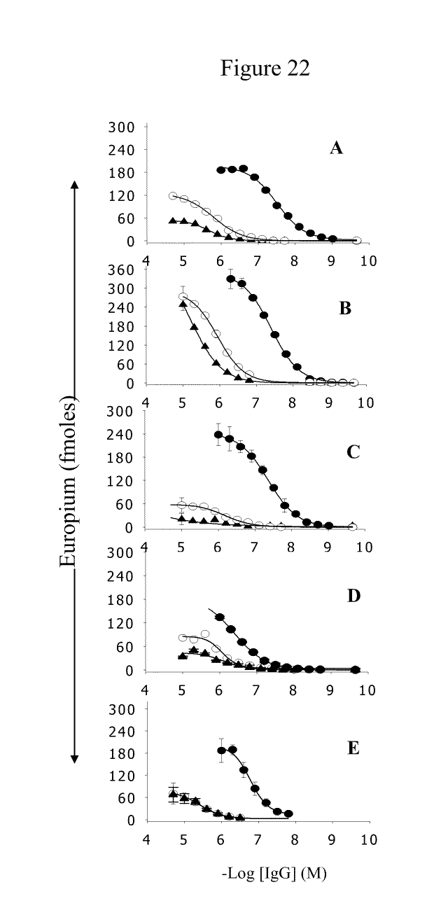

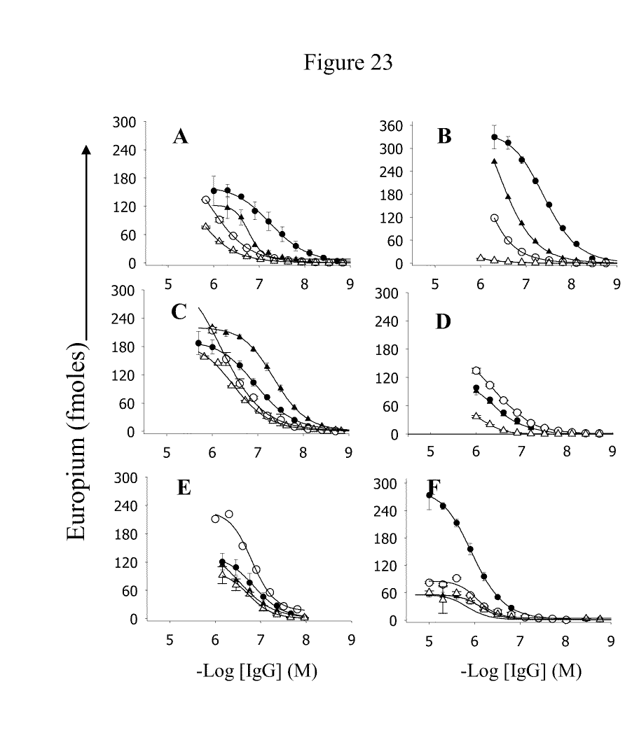

Naturally occurring Aβ oligomer- and fibril-reactive antibodies bind to the same fibril-related epitope(s): An affinity column was used to isolate Aβ oligomer-reactive antibodies in IGIV in which Sepharose beads were conjugated with CAPS generated from the Aβ40 peptide. Based on the protein concentrations of the filtrate and eluate, the recovered oligomer-reactive antibodies represented ˜0.1% of the immune globulin passed through the column, i.e., ˜5 mg from a bottle containing 5 g of IGIV. FIGS. 22 and 23 show that the affinity purified antibodies were ˜50-fold stronger at binding to Aβ oligomers than a native IGIV preparation, and these molecules bound similarly to Aβ fibrils and oligomers, but weakly to monomer.

Similarly, JTO fibril affinity purified IGIV bound equally to Aβ fibrils and oligomers (FIGS. 22 & 23), suggesting that the same subgroup of antibodies was eluted off the fibril or Aβ oligomer affinity column. Further affinity chromatography experiments, which involved depleting IGIV of Aβ oligomer- or fibril-reactivity, resulted in a loss of IGIV binding to amyloid fibrils and Aβ oligomers. These results again showed that there is a diverse subpopulation of naturally occurring human antibodies in IGIV that cross-react with Aβ fibrils and oligomers. Presumably, these antibodies bind to common fibril-related conformational epitope(s) since we have shown that there were no antibodies in IGIV that were specific (or at least at a high concentration) against the Aβ sequence.

Cross-linked Aβ oligomers are a potent immunogen: Active vaccination with Aβ40 oligomers elicited a strong antibody response in mice, with saturated antibody binding observed even after a 1:13,000 sera dilution. Hybridoma fusion of B-cells from the spleen of one of these mice resulted in several stable cell clones that produce anti-Ap oligomer antibodies.

### Example 3

Therapeutic studies indicate that active or passive vaccination against amyloid fibrils or prefibrillar conformers is a feasible curative strategy for patients with amyloid associated diseases. The inventors have recently used fibril chromatography to show that human sera contain a subpopulation of fibril-reactive IgGs having therapeutic and diagnostic potential for patients with Alzheimer's disease or other amyloidoses (O'Nuallain et al, 2006). In this Example, the inventors show that Aβ-reactive antibodies contained in normal human sera are directed against a limited number of common conformational epitopes on A, oligomers and fibrils with little or no binding to the monomer precursor peptide per se.

Materials and Methods

Peptides, Proteins, and Antibodies. Human IAPP, wild-type Aβ40 and Aβ42, F19P Aβ40, and N- and C-terminal cysteinylated Aβ40 were purchased from Quality Controlled Biochemicals (Hopkinton, Mass.). The peptide preparations were >90% pure, as determined by standard mass spectrometric (MS) analysis. Before use, each lyophilized Aβ40 peptide was disaggregated by sequential exposure to trifluoroacetic acid (TFA) and hexafluoroisopropanol (HFIP), 2 mM NaOH added and 2×PBS added to 1× to give a final peptide concentration of ˜0.2 mg/ml, as previously described (O'Nuallain et al. 2002). Alternatively, the peptide was prepared by alkaline pretreatment (Fezoui et al. 2000) that involved solvating the peptide at ˜1 mg/ml in 2 mM NaOH for ˜5 min., 2×PBS added to 1×, a 3 min. sonication on ice, using a probe sonic disruptor (Teledyne/Tekmar), followed by centrifugation at 20,800×g for 30 min at 4° C. Soluble Aβ42 peptide was prepared using a modified version of Teplow's alkaline pretreatment protocol (Teplow 2006). Briefly, the peptide was disaggregated by TFA HFIP (O'Nuallain et al. 2002), and 75 μl of ˜1 mg/ml peptide in HFIP sample (˜75 μg Aβ) was added into glass tubes. The samples were evaporated under argon, lyophilized for 1 h, and 1 ml 2 mM NaOH added. Samples were then pooled into 4 ml amounts, snap-frozen (liquid nitrogen), and lyophilized overnight. PBS (4 ml of 1×) was added to each sample (˜0.04 mg/ml), transferred to polycarbonate ultracentrifuge tubes, and centrifuged at 302,000×g for 1 h at 4° C. Peptide concentration was determined at A₂₁₅ₙₘ by reverse-phase HPLC using a C3 reverse-phase Zorbax column (Agilent) and a standard curve calibrated from an Aβ40 stock whose concentration was determined by amino acid composition analysis (Kheterpal et al. 2001; O'Nuallain et al. 200, 2004). Human IAPP was solubilized and disaggregated using a 1:1 mixture of TFAIHFIP as previously described (Kheterpal et al. 2001). Briefly, after removal of volatile solvents, the peptide was dissolved in 2 mM NaOH and centrifuged at 20,800×g for 25 min. The supernatant was diluted 1:2 by using a 2×PBS stock containing 0.1% sodium azide, pH 7.4, to a final concentration of 0.25 mg/ml.

Recombinant (r) V_{λ}6 Jto was produced in E. coli, as previously described (Wall et al. 1999). The lyophilized protein was dissolved in distilled water to a concentration of ˜1 mg/ml (˜80 μM) and 10×PBS containing 0.5% sodium azide added to 1× (PBSA), and the sample passed through a 0.22 μm PVDF 25 mm Millex®-GV syringe-driven filter unit (Millipore, Beillerica, Mass.). Protein concentration was determined spectrophotometrically, using 13,490 M⁻¹ cm⁻¹ as E₂₈₀ (http://helix.nih.gov/docs/gcg/peptidesort.html), and the resulting preparation aliquoted and stored at −20° C.

Chicken egg white ovalbumin and lysozyme were purchased from Sigma. IGIV (Gammagard Liquid®) was provided by Baxter AG/Biosciences. A monoclonal antibody (mAb) against the N-terminus of Aβ (MAB 1560) was from Chemicon (Temecula, Calif.).

Preparation of CAPS: CAPS were prepared from high pH pretreated Aβ40 (˜0.2 mg/ml) and Aβ42 (˜0.05 mg/ml) by incubating the peptides with 1.1 μM HRP and 250 μM H₂O₂ in PBS at 37° C. for 3 h. CAPS were partially purified by adding 1 mM CuSO₄ copper [10], incubating the sample for 2 h at room temperature followed by centrifugation at 20,800×g for 30 min, and removal of the supernatant. Immediately, 3 M guanidine-HCl was added and the pellet resuspended and incubated for 30 min. at room temperature to remove any bound HRP, and centrifuged, as before. The pellet was resuspended again, followed by 3×PBS washes, and CAPS were resolubilized to a final concentration of ˜0.2 mg/ml by the addition of 5 mM EDTA in PBS for 2 h at room temperature followed by the removal of any insoluble aggregates by centrifugation. The preparation was dialyzed, using a 5000 MW cut-off membrane (Fisher), centrifuged, as before, and used immediately or snap frozen (liquid N₂) and stored at −80° C. for up to 1 mo. Quantification of the soluble reaction product was carried out using SDS PAGE (4-12% Bis Tris precast gels; Invitrogen Corp.) and the MicroBCA assay (Pierce). Electrospray ionization mass spectrometry (Applied biosystems (Foster City, Calif.)), and dityrosine fluorescence (Malencik et al. 2003) confirmed that the aggregates consisted of low molecular weight (<38 kDa) cross-linked SDS stable species.

Preparation of noncovalent Aβ42 and lysozyme oligomers, and prefibrillar IAPP aggregates: SDS stable Aβ42 oligomers and lysozyme oligomers were prepared as described previously (Barghorn et al. 2005; Gharibyan et al. 2007). Prefibrillar IAPP aggregates were prepared by incubating the TFA/HFIP pretreated peptide in PBS at ˜0.2 mg/ml for 5 h at 37° C., as described previously (O'Nuallain et al. 2004).

Preparation of Amyloid Fibrils: Aβ40 fibrils, prepared from the TFA/HFIP disaggregated peptide, and Jto fibrils were prepared and reaction monitored by thioflavin T as described previously (O'Nuallain et al. 2002). All fibril samples were harvested by centrifugation, 20,200×g for 30 min at room temperature, sonicated (2×30 s bursts) with a probe sonicator disruptor (Teledyne/Tekmar), aliquoted, and stored at −20° C.

Preparation of Aβ Conformer and Jto Fibril Affinity Columns: Each Aβ40 conformer (sonicated fibrils, CAPS, or monomer) was linked covalently to an N-hydroxysuccinimide (NHS)-activated Sepharose® 4 fast-flow pre-activated agarose matrix with a mean bead size of 90 μm (Amersham Biosciences Corp., Piscataway, N.J.). For this procedure, 1 mg/ml of conjugate in PBS per 1 ml of packed bed volume of matrix was gently mixed at room temperature for 3 h. The final product was poured into a plastic polypropylene column (Pierce) and equilibrated with PBS. A monomeric F19P mutant Aβ40 peptide column was prepared by gently mixing 1 mg ml equimolar mix of N- and C-terminal cysteinylated Aβ mutant peptides in 50 mM Tris, 5 mM EDTA, pH 8.5 per ml of packed bed volume of iodoacetyl coupling gel (SulfoLink coupling resin; Pierce) for 45 mim at room temperature. The resin was deactivated using L-cysteine and the column equilibrated with PBS as per manufacturers recommendations. Reverse phase HPLC showed that >80% of the Aβ conformers conjugated to the NHS-activated matrix and 50% of the F19P Aβ peptides conjugated to the iodoacetyl gel.

Preparation of Affinity-Purified Antibody: IGIV was Filtered to Render the Preparation aggregate-free, diluted with PBS to yield a final concentration of 10-20 mg/ml, and loaded onto the appropriate Jto fibril, Aβ40 fibril or CAPS column that were pre-equilibrated with PBS. to To maximize peptide accessibility, and before loading the antibody preparation, wild-type and F19P mutant Aβ monomer columns were prewashed with 2 column volumes of 6 M guanidine-HCl followed by 2× washes with PBS. Any weakly bound IgG was removed with 40 ml of PBS and column-bound antibodies eluted in 1-ml portions using 0.1 M glycine buffer, pH 2.7, and fractions neutralized by addition of 1 M Tris HCl, pH 9. The concentration of IgG in the affinity-purified eluents and residual samples was determined based on absorbance at 280 nm, using an E₂₈₀^{1%} of 1.30 and a mol wt of 150000 daltons. Samples containing the enriched antibodies were pooled and concentrated with a PL-30 Centricon® (Millipore) apparatus and stored at 4° C. for ˜1 wk, to remove the transiently-induced Aβ-reactivity that occurred on exposure of the antibodies to low pH eluting buffer (Li et al. 2007). Long term storage was at ˜20° C.

Preparation of F(ab′) antibody fragment. F(ab′) antibody fragment was prepared using a Fab preparation kit as per manufacturers procedure (Pierce, Rockford, Ill.; cat#44885). Briefly, this involved digestion of 4 mg/ml human IgG by agarose-immobilized papain for 4 h at 37° C., followed by separation of the F(ab′) reaction product by passing the reaction mixture over a protein A column.

Antibody-Binding Microtiter Plate Assay: The relative strength of antibody binding with Aβ conformers, Jto fibrils, and control proteins was determined by a europium (Eu³⁺)-based fluoroimmunoassay (EuLISA) (Diamandis 1988; O'Nuallain et al. 2007). All measurements in this and other assays were done in triplicate (error bars in the figures represent SD). Briefly, human plasma (provided by Baxter AG/Biosciences), MAB 1560, or IgG fractions were serially diluted in activated, high-binding microtiter plate wells (COSTAR, Corning, N.Y.) that were directly coated with 400-500 ng of protein and blocked with 1% BSA in PBS. Alternatively, binding studies were carried out against covalently attached protein via poly-L lysine/glutaraldehyde attachment (Kennel 1982). For competition studies, the concentration of antibody (50-80 nM) and inhibitors (˜0.2 mg/ml) remained constant. A biotinylated goat anti-human IgG (γ-chain specific, Sigma) or biotinylated goat anti-mouse IgG reagent served as secondary antibody and, after addition of a Eu³⁺-streptavidin conjugate followed by the releasing enhancement solution, Eu³⁺ time-resolved fluorescence was measured with a Perkin Elmer Victor² 1420 Multilabel Counter. The amount (N) of lanthanide released was calculated from a standard curve using known concentrations of Eu³⁺. All measurements in this and other assays were done in triplicate (error bars in the figures represent SD). Values for EC₅₀ and IC₅₀ were determined from sigmoidally fit antibody binding curves (SigmaPlot 2000 ver. 6; Systat Software, Inc.).

Western blot analysis of CAPS binding: CAPS bands were transferred from 4-12% Bis-tris gels (Invitrogen) onto PVDF transfer membranes (0.2 μm pore size; Invitrogen) using NuPAGE transfer buffer (Invitrogen) and 30 V for 1 h. The membranes were blocked with 1% BSA in PBS, 100 nM of anti-Aβ conformer IGIV, or MAB1560 added, 3×PBS containing 0.05% tween 20 washes, and goat anti-human or mouse IgG conjugated to HRP added. After washing, antibody-binding was detected using a 3,3′-diaminodbenzidine (DAB) substrate kit (Pierce).

Results

Human IgGs bind to a limited number of common conformational epitopes on LC fibrils, Aβ fibrils and CAPS: To systematically determine which Aβ species the human immune response is directed against, antibody fractions were isolated off LC fibrils and Aβ40 conformer columns as shown in FIG. 22. Antibody isolated off LC fibril, and Aβ40 conformer columns bound 2-50 times stronger than unfractionated IGIV to the plate-immobilized amyloidogenic conformer that was used for purification. The most enriched preparations were obtained using LC and Aβ40 fibril affinity chromatography, with ˜50- and 30-fold enrichment, respectively, followed by ˜20- and 10-fold enhancement using CAPS and monomer columns, respectively (FIG. 22 and Table 3). EC₅₀ values for binding to the wild-type conformer used for isolation ranged from ˜40 nM for LC fibrils, Aβ40 fibrils and CAPS, to ˜300 nM for monomer binding (FIG. 22 and Table 3). Notably, fractionation resulted in a 2-4 fold increase in the maximum signal amplitude for LC fibril and Aβ40 conformer binding (FIG. 22). To ensure that the relatively small enrichment and low affinity antibody off the Aβ40 monomer column was not due to a lack of peptide accessibility as a result of peptide immobilization, Aβ-reactive IGIV was also isolated using a column consisting of an equimolar mixture of immobilized N- and C-terminal cysteinylated mutant F19P peptide. This peptide is less prone to aggregation than wild-type Aβ40 (Bernstein et al. 2005; Cannon et al. 2004). Antibody eluted off the F19P peptide column bound with similar affinity to plate-immobilized wild-type and F19P Aβ40 peptide, and, notably, reactivity with the wild-type peptide was 2-fold stronger, EC₅₀ value of 157 nM, than that with wild-type Aβ40 monomer purified IgG, EC₅₀ value of ˜352 nM (FIGS. 22 & 23, Table 4).

TABLE 3
Comparison of EC₅₀ values and maximum signal amplitudes for Aβ40 conformer-reactive
unfractionated, residual, and enriched IGIV preparations.

| | Unfractionated IgG (unfr.) | | Residual (res.) | | Enriched (enr.) | | | |
|---|---|---|---|---|---|---|---|---|
| | EC₅₀ | Max. signal | EC₅₀ | Max. signal | EC₅₀ | Max. signal | EC₅₀ | Max. signal |
| Column | (nM) | amplitude | (nM) | amplitude | (nM) | amplitude | (unfr. enr.) | (unf. enr.) |
| LC fibril | 1514 ± 3 | 120 ± 0.2 | 2113 ± 2 | 53.3 ± 0.1 | 30.6 ± 0.1 | 194 ± 0.1 | 50.5 | 0.62 |
| Aβ40 | 1084 ± 1 | 283 ± 0.3 | 4508 ± 18 | 336 ± 1 | 38.5 ± 0.1 | 339 ± 0.1 | 28.2 | 0.83 |
| fibril | | | | | | | | |
| CAPS | 631 ± 0.1 | 58.5 ± 0.1 | ~8000 | ~75 | 42.9 ± 0.1 | 238 ± 0.2 | 14.7 | 0.25 |
| Wild-type | 861 ± 0.1 | 85.7 ± 0.1 | 753 ± 0.1 | 43.8 ± 0.1 | 352 ± 1 | 175 ± 0.3 | 2.45 | 0.49 |
| Aβ40 mon | | | | | | | | |
| F19P | 3673 ± 0.1 | 75.4 ± 0.1 | 3184 ± 32 | 69.7 ± 4 | 163 ± 0.1 | 199 ± 0.1 | 22.5 | 0.38 |
| Aβ40 mon | | | | | | | | |

TABLE 4
Comparison of EC₅₀ values and maximum signal amplitudes for Aβ40 conformer-reactive
IgGs isolated off LC fibril and Aβ40 conformer columns

| | Fibril | | CAPS | | Wild-type or F19P monomer | |
|---|---|---|---|---|---|---|
| Column | EC₅₀ | Max. signal | EC₅₀ | Max. signal | EC₅₀ | Max. signal |
| or IGIV | (nM) | amplitude | (nM) | amplitude | (nM) | amplitude |
| LC fibril | 53 ± 0.1 | 161 ± 0.0 | 170 ± 0.0 | 122 ± 0.1 | ~1000 | ~200 |
| | | | | | (~500) | (~200) |
| Aβ40 fibril | 31 ± 0.0 | 339 ± 0.1 | ~300 | ~400 | ~1000 | ~400 |
| | | | | | (~10000) | (n.d.) |
| CAPS | 109 ± 0.2 | 197 ± 0.3 | 42.9 ± 0.1 | 238 ± 0.2 | 713 ± 0.3 | 369 ± 1.1 |
| | | | | | (353 ± 1) | (198 ± 0.4) |
| Wild-type | ~1000 | ~150 | ~1000 | ~150 | 352 ± 1 | ~300 |
| Aβ40 mon | | | | | (~5000) | (n.d.) |
| F19P Aβ40 | 141 ± 0.0 | 136 ± 0.1 | 307 ± 0.0 | 158 ± 0.2 | 157 ± 0.1 | 229 ± 0.1 |
| mon | | | | | (163 ± 0.1) | (150 ± 0.1) |
| IGIV | 1084 ± 1 | 283 ± 0.3 | 631 ± 0.1 | 58.5 ± 0.1 | 861 ± 0.1 | 85.7 ± 0.1 |
| | | | | | (3673 ± 0.1) | (75.4 ± 0.1) |

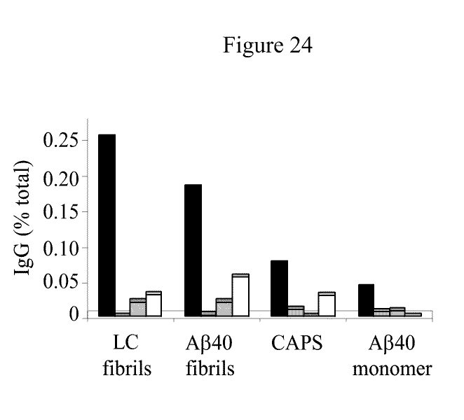

Determination of antibody binding curves against Aβ40 conformers for each LC fibril-, Aβ40 fibril-, or CAPS-purified antibody preparation showed that preferential binding was against the conformer used for isolation, with 2-5 fold weaker binding to other aggregated Aβ40 species, and up to ˜30-fold weaker interactions with the monomeric peptide (FIG. 23 & Table 4). In contrast with antibodies isolated using amyloidogenic aggregates, wild-type and cysteinylated F19P Aβ40 monomer-purified IgGs resembled unfractionated IGIV, in binding similarly to immobilized Aβ40 fibrils, CAPS, and monomers, with EC₅₀ values of ˜250 nM and ˜150 nM for the two antibody preparations, respectively (FIG. 23 and Table 4). Five- and 10-fold weaker F19P mutant Aβ40-binding was observed for LC fibril, and Aβ40 fibril and wild-type monomer isolated antibodies compared with their affinity for wild-type Aβ40 monomer, indicating the importance of phenylalanine at position 19 for antibody interactions (FIG. 23 & Table 4). Remarkably, although antibodies eluted off LC fibril and Aβ conformer columns after one passage of IGIV had diverse Aβ conformer binding properties, several passages of IGIV over any one of these columns, until binding is essentially depleted, resulted in homogenous preparations that accounted for 0.3% of total antibodies in IGIV, and almost complete loss of IGIV reactivity against LC fibrils and all three Aβ40 conformers (FIG. 24).

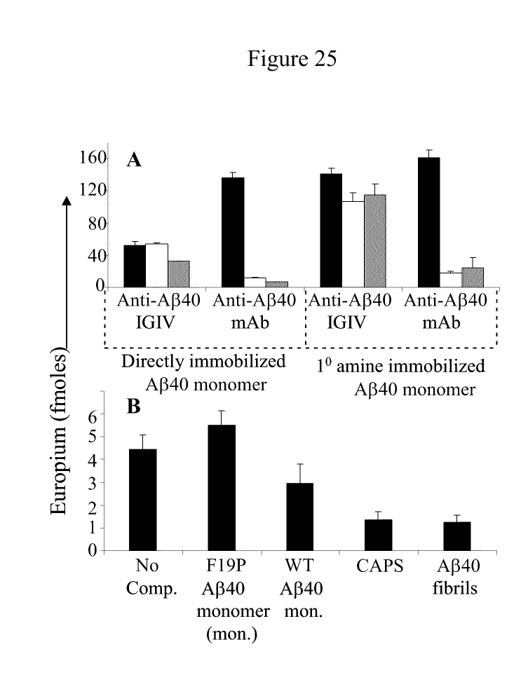

Aβ40 monomer affinity-isolated antibodies bind to cryptic epitopes on LC fibrils, Aβ fibrils, CAPS, and surface-adsorbed Aβ monomer: Aβ competition studies, using intact and a F(ab′) fragment of wild-type Aβ40 monomer-isolated IgGs as well as a anti-Aβ antibody control (MAB1560; Chemicon) were carried out to determine whether Aβ conformer-purified antibody binding to plate-immobilized Aβ40 monomer was against an epitope that was only exposed on plate-adsorption. FIG. 25 shows that a 100-fold molar excess of wild-type or F19P Aβ40 monomer was unable to prevent Aβ40 monomer-isolated antibody binding to the monomeric peptide directly coated or immobilized using poly-L-lysine/glutaraldehyde chemistry. In contrast, the monomeric wild-type and F19P Aβ peptides were potent inhibitors of an anti-Aβ mAb, MAB 1560, which bound to an N-terminal epitope (FIG. 25A). The inability of Aβ monomer-isolated IGIV to react with solution-phase Aβ40 monomers (relative to the plate-immobilized peptide) was not due to lower avidity as evidenced by the weak inhibition observed with the wild-type monomeric peptide and the inability of the F19P mutant peptide to prevent a F(ab′) fragment binding to the immobilized wild-type peptide (FIG. 25B). Notably, although cross-linked Aβ oligomers and fibrils have less accessible peptides available for binding, these conformers were twice as potent as wild-type Aβ40 monomer for inhibiting F(ab′) binding. This indicated that reactivity against the plate-immobilized monomeric peptide is not directed against the peptide's sequence per se, but at a surface-induced conformational entity that is also present on fibrils and CAPS.

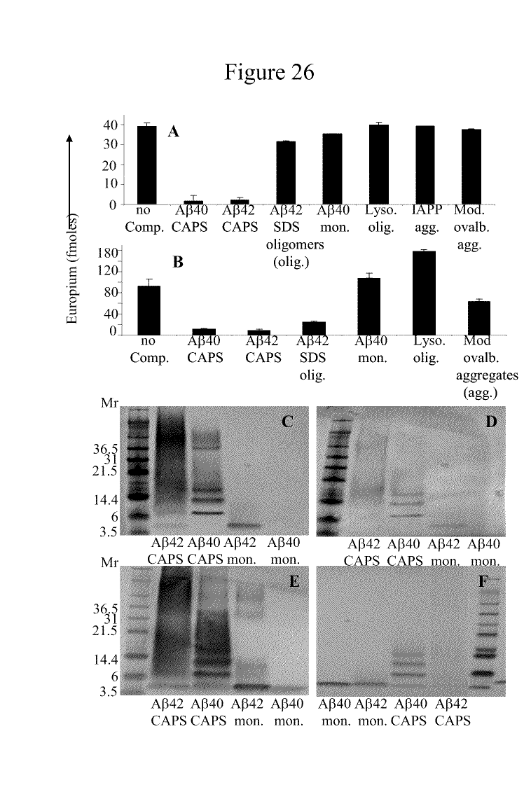

Fibril and CAPS isolated antibodies have diverse Aβ conformer-reactivity: Competition studies, Aβ conformer-reactivity in the presence and absence of human plasma, and Western blot analysis were carried out to further characterize the binding properties of Aβ fibril and CAPS-isolated antibodies. FIG. 26A shows that binding of CAPS-isolated IgGs to plate-immobilized CAPS (consisting of Aβ40), was almost completely inhibited by a 50-fold molar excess of solution-phase CAPS (both Aβ40 and Aβ42 species). Only weak competition was evident with the same amount of non-covalent Aβ42 SDS stable oligomers, and little or no inhibition was apparent with lysozyme oligomers, prefibrillar IAPP aggregates, non-amyloidogenic reduced and alkylated ovalbumin aggreates, and Aβ40 monomers (FIG. 26A). In contrast, both CAPS and Aβ42 SDS stable oligomers were similarly potent inhibitors of fibril-isolated antibody binding to plate-immobilized CAPS, and reduced and non-alkylated ovalbumin aggregates also had activity, albeit half as weak as the Aβ conformers (FIG. 26B). Western blot analysis confirmed our EuLISA results, in that CAPS- and Aβ40 fibril-isolated antibodies selectively bound to the aggregated peptide, with a smear of reactivity against CAPS prepared from Aβ42, but more discrete dimer, trimer, tetramer, and decamer (only evident with CAPS-isolated antibody) peptide bands were stained with CAPS prepared using Aβ40 (FIGS. 26C, D). Notably, the antibodies did not stain the Aβ40 monomer band but showed some activity against the more conformation prone Aβ42 peptide. A commercial N-terminal anti-Aβ mAb, MAB 1560, did not stain the decamer peptide band at ˜40 kDa that the anti-CAPS preparation bound to, and this antibody had somewhat different staining patter than either the CAPS or fibril-isolated antibodies (FIGS. 26C-F).

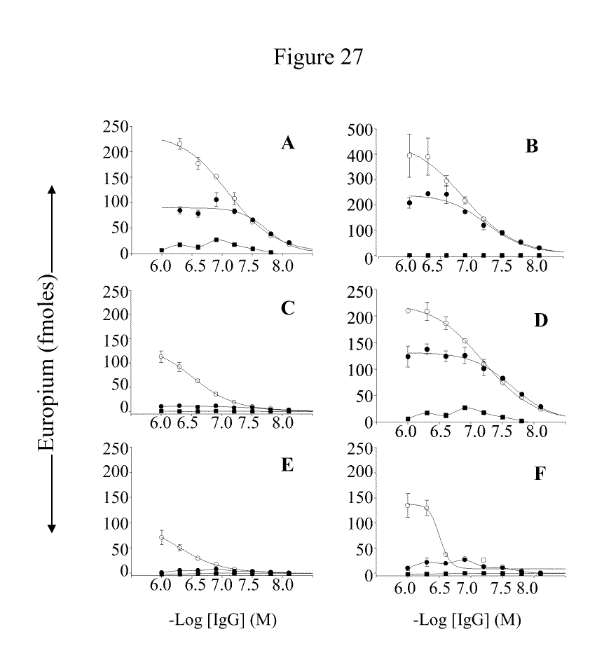

The diverse Aβ conformer reactivity of Aβ40 fibril- and CAPS-isolated antibody preparations was also evident from binding studies carried out in the presence and absence of human plasma. FIG. 27 shows that CAPS-purified antibodies retained more reactivity than fibril-isolated antibody against Aβ40 fibrils and CAPS. Plasma reduced the maximum binding signal amplitudes for antibody binding to Aβ40 fibrils by ˜half (FIGS. 27A, B & Table 5). Similarly, plasma reduced the maximum signal amplitude by half for CAPS-isolated antibody binding to CAPS, but ˜20-fold decrease in signal was observed for the anti-fibril enriched antibody preparation. However, the addition of plasma resulted in a 3- and up to a 10-fold increase in CAPS and fibril-isolated antibody binding affinity for Aβ conformer, respectively, with EC₅₀ values of ˜18-50 nM (FIG. 27 and Table 5). A similar plasma effect was observed for antibody binding to Aβ40 monomer, however, with each antibody preparation, maximum binding signal amplitude was drastically reduced by up to 50-fold, giving pitiful binding signal compared to that obtained with Aβ40 fibrils (FIG. 27 & Table 4).

TABLE 5
Effect of human plasma on EC₅₀ values and maximum signal amplitudes for anti-Aβ conformer
IgGs isolated using Aβ40 fibril or CAPS column.

| | Anti-Fibril enriched | | | | Anti-CAPS enriched | | | |
|---|---|---|---|---|---|---|---|---|
| | | | +plasma | | | | +plasma | |
| Aβ40 | EC₅₀ | Max. signal | EC₅₀ | Max. signal | EC₅₀ | Max. signal | EC₅₀ | Max. signal |
| conformer | (nM) | amplitude | (nM) | amplitude | (nM) | amplitude | (nM) | amplitude |
| Fibril | 49.2 ± 0.0 | 238 ± 0.2 | 17.6 ± 0.1 | 90.2 ± 0.2 | 134 ± 1.3 | 461 ± 36 | 54.7 ± 0.1 | 240 ± 21 |
| CAPS | 224 ± 0.0 | 226 ± 0.1 | 21.9 ± 0.0 | 10.1 ± 1 | 63.2 ± 0.1 | 226 ± 0.1 | 21.9 ± 0.0 | 132 ± 0.1 |
| Monomer | 547 ± 0.1 | 106 ± 0.2 | ~30 | ~10 | 138 ± 0.02 | 313 ± 0.0 | ~40 | ~20 |

Discussion

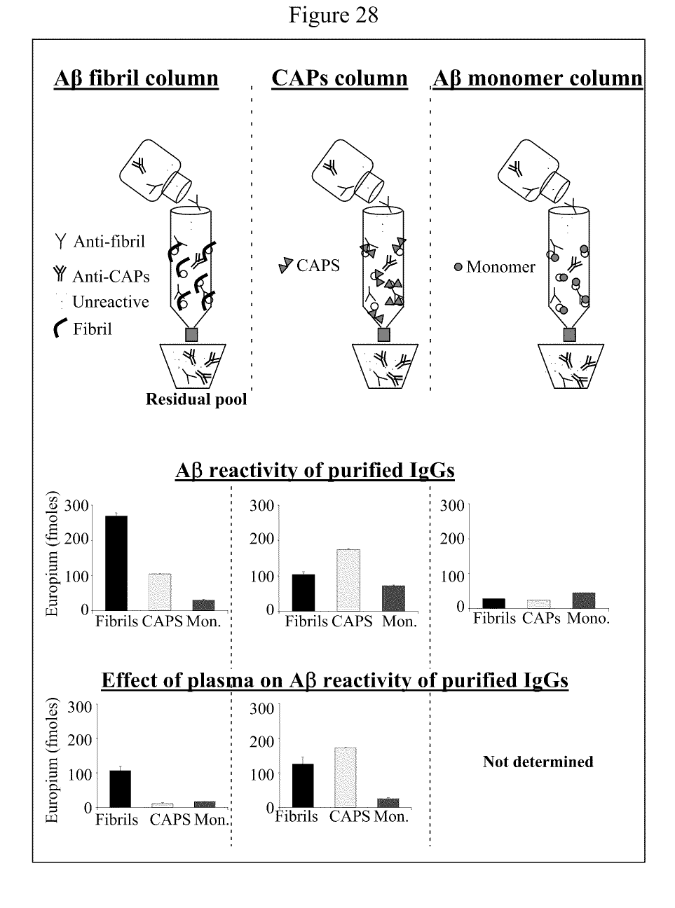

The results provide conclusive evidence that Aβ-reactive antibodies contained in normal human sera are directed against a limited number of common conformational epitopes on Aβ oligomers and fibrils with little or no binding to the native solution-phase monomer precursor peptide per se. Any in vivo reactivity against the native Aβ peptide or its unbiquitous transmembrane precursor protein, APP, would likely be detrimental, given that there is experimental evidence that these molecules are involved in cholesterol and lipid homeostasis as well as memory and neural differentiation (Heese et al. 2006; Senechal et al. 2007; Kwak et al. 2006; Priller et al. 2006). Furthermore, our studies show that although antibody preparations isolated off Aβ fibril and CAPS columns each contain antibodies that bind to common conformational epitopes on LC fibrils, Aβ fibrils, and CAPS, these fractions contain distinct Aβ conformer reactivity. This was evidenced from results obtained from our competition studies, antibody affinities, and antibody experiments carried out in the presence of human plasma. FIG. 28 shows a schematic of the most imperative results obtained from these studies.

It should be understood that the foregoing discussion and examples merely present a detailed description of certain preferred embodiments. It therefore should be apparent to those of ordinary skill in the art that various modifications and equivalents can be made without departing from the spirit and scope of the invention. All journal articles, other references, patents, and patent applications that are identified in this patent application are incorporated by reference in their entirety.

### REFERENCES

ABC. (2005) Antibodies may help Alzheimer's: study. http://abc.com.au/news/stories/2005/04/13/1344116.htm.
Ali et al., Dimerisation of N-acetyl-L-tyrosine ethyl ester and Abeta peptides via formation of dityrosine. Free Radic. Res., 40(1): 1-9 (2006).
Amado et al., Dityrosine: in vitro production and characterization, Methods Ebzymol., 107: 377-88 (1984).
Atwood et al., Copper-Mediated Aggregation And Polymerization of Aβ, Soc. Neurosci. Abstr., 23: 1883 (1997).
Atwood, C. S., et al., Copper mediates dityrosine cross-linking of Alzheimer's amyloid-beta, Biochemistry, 43(2): 560-8 (2004). B. O'N. and A. S.; unpublished results.
Barghom et al., Globular amyloid beta-peptide oligomer—a homogenous and stable neuropathological protein in Alzheimer's disease, Journal of neurochemistry, 95(3): 834-7 (2005).
Benson M D. Amyloidosis. The Metabolic and Molecular Bases of Inherited Disease: In: Beaud et al, Sly W S and Valle D (eds.) 8th ed., 5345-78 (2001).
Bernstein et al., Amyloid beta-protein: monomer structure and early aggregation states of Abeta42 and its Pro19 alloform, Journal of the American Chemical Society, 127(7): 2075-84 (2005).
Cannon et al., Kinetic analysis of beta-amyloid fibril elongation, Anal. Biochem., 328(1): 67-75 (2004).
Chiti et al., Protein misfolding, functional amyloid, and human disease, Annu. Rev. Biochem., 75: 333-66 (2006)
Clackson et al., Making antibody fragments using phage display libraries, Nature, 352(6336): 624-28 (1991).
Clackson, Genetically engineered monoclonal antibodies, Br. J. Rheumatol., 2: 36-9 (1991).
Corder et al., Gene dose of apolipoprotein E type 4 allele and the risk of Alzheimer's disease in late onset families, Science, 261(5123): 921-3 (1993).
Curin-Serbec et al. Monoclonal antibody against a peptide of human prion protein discriminates between Creutzfeldt-Jacob's disease-affected and normal brain tissue, J. Biol. Chem., 279: 3694-8 (2004).
Diamandis, E. P. Immunoassays with time-resolved fluorescence spectroscopy: principles and applications, Clin. Biochem., 21(3): 139-50 (1988).
Dodel et al., Intravenous immunoglobulins containing antibodies against beta-amyloid for the treatment of Alzheimer's disease, Journal of Neurology, Neurosurgery, and Psychiatry, 75(10): 1472-4 (2004).
Du et al, Human anti-β-amyloid antibodies block β-amyloid fibril formation and prevent β-amyloid-induced neurotoxicity, Brain, 126(9): 1935-9 (2003).
Dumoulin et al, Probing the origins, diagnosis and treatment of amyloid diseases using antibodies, Biochimie, 86:589-600 (2004).
Enqvist et al., Senile amyloidoses—diseases of increasing importance, Acta Histochemica, 105(4): 377-8 (2003).
Enqvist et al., Senile amyloidoses—diseases of increasing importance, Acta histochemica, 105(4): 377-8 (2003).
Fezoui et al., An improved method of preparing the amyloid beta-protein for fibrillogenesis and neurotoxicity experiments, Amyloid, 7(3): 166-78 (2000).
Franklin et al., Antisera specific for human amyloid reactive with conformational antigens, Proc. Soc. Exp. Biol. Med., 140(2): 565-8 (1972).
Galeazzi et al. In vitro peroxidase oxidation induces stable dimers of beta-amyloid (1-42) through dityrosine bridge formation, Amyloid, 6(1): 7-13 (1999).
Gaskin et al., Human antibodies reactive with β-amyloid protein in Alzheimer's disease, J. Exp. Med., 177: 1181-6 (1993).
Gevorkian et al., Mimotopes of conformational epitopes in fibrillar β-amyloid, J. Neuroimmunol., 156:10-20 (2004).
Gharibyan et al., Lysozyme amyloid oligomers and fibrils induce cellular death via different apoptotic/necrotic pathways, J. Mol. Biol., 365(5): 1337-1349 (2007).
Glabe, C. G. Conformation-dependent antibodies target diseases of protein misfolding, Trends Biochem. Sci., 29(10): 542-7 (2004).
Goedert et al., A century of Alzheimer's disease, Science, 314(5800): 777-81 (2006).
Golde et al., The Abeta hypothesis: leading us to rationally-designed therapeutic strategies for the treatment or prevention of Alzheimer disease, Brain Pathol., 15(1): 84-7 (2005).
Goldsbury et al., Polymorphic fibrillar assembly of human amylin, J. Struct. Biol., 119: 17-27 (1997).
Goldsbury et al., Time-lapse atomic force microscopy in the characterization of amyloid-like fibril assembly and oligomeric intermediates. Methods Mol. Biol., 299: 103-28 (2005).
Goldsteins et al., Exposure of cryptic epitopes on transthyretin only in amyloid and in amyloidogenic mutants. Proc. Natl. Acad. Sci. USA, 96: 3108-13 (1999).
Gross et al., The oxidation of tyramine, tyrosine, and related compounds by peroxidase, J. Biol. Chem., 234: 1611-4 (1959).
Harding et al., Non-enzymic post-translational modification of proteins in aging, A review. Mech. Ageing Dev., 50(1): 7-16 (1989).
Hardy et al., The amyloid hypothesis of Alzheimer's disease: progress and problems on the road to therapeutics. Science, 297(5580): 353-6 (2002).
Hardy et al., The amyloid hypothesis of Alzheimer's disease: progress and problems on the road to therapeutics, Science, 297(5580): 353-6 (2002).
Harlow et al. Antibodies: a laboratory manual, Cold Spring Harbor Press, 1988.
Heese et al., Alzheimer's disease—an interactive perspective, Current Alzheimer research, 3(2): 109-21 (2006).
Heineck et al., Tyrosyl radical generated by myeloperoxidase catalyzes the oxidative cross-linking of proteins, J. Clin. Invest., 91(6): 2866-72 (1993).
Henkel et al., Immune complexes of auto-antibodies against A1-42 peptides patrol cerebrospinal fluid of non-Alzheimer's patients, Molecular psychiatry, 12(6): 601-10 (2007).
Hensley et al., Electrochemical Analysis of Protein Nitrotyrosine and Dityrosine in the Alzheimer Brain Indicates Region-Specific Accumulation, J. Neurosci., 18: 8126-32 (1998).
Hrncic et al., Antibody-mediated resolution of light chain-associated amyloid deposits. Am. J. Pathol., 157: 1239-46 (2000).
Hull et al., Disease-Modifying Therapies in Alzheimer's Disease: How Far Have We Come? Drugs, 66(16): 2075-93 (2006).
Istrin et al., Intravenous immunoglobulin enhances the clearance of fibrillar amyloid-beta peptide, J. Neurosci. Res., 84(2): 434-43 (2006).
Jacob et al., Human Phagocytes Employ the Myeloperoxidase-Hydrogen Peroxide System to Synthesize Dityrosine, Trityrosine, Pulcherosine, and Isodityrosine by a Tyrosyl Radical-dependent Pathway, J. Biol. Chem., 271(33): 19950-6 (1996).
Kayed et al., Common structure of soluble amyloid oligomers implies common mechanism of pathogenesis. Science, 300: 486-9 (2003).
Kennel, S. J., Binding of monoclonal antibody to protein antigen in fluid phase or bound to solid supports, Journal of immunological methods, 55(1): 1-12 (1982).
Kheterpal et al., Structural features of the Abeta amyloid fibril elucidated by limited proteolysis, Biochemistry, 40(39): 11757-67 (2001).
Klyubin et al., Active and passive immunotherapy for Alzheimer's disease: direct neutralization of in vivo synaptic plasticity disrupting amyloid β-protein oligomers, Nature medicine, 11(5): 556-61 (2005).
Kohler et al., Continuous cultures of fused cells secreting antibody of predetermined specificity, Nature, 256: 495-7 (1975).
Kwak et al., Amyloid precursor protein regulates differentiation of human neural stem cells, Stem cells and development, 15(3): 381-9 (2006).
Landsbury et al., Structural model for the beta-amyloid fibril based on interstrand alignment of an antiparallel-sheet comprising a C-terminal peptide. Nat. Struct. Biol., 2: 990-8 (1995).
Lee et al., Targeting amyloid-beta peptide (Abeta) oligomers by passive immunization with a conformation-selective monoclonal antibody improves learning and memory in Abeta precursor protein (APP) transgenic mice, J. Biol. Chem., 281(7): 4292-9 (2006).
Lee, V. M., Abeta immunization: moving Abeta peptide from brain to blood, Proc. Natl. Acad. Sci., USA, 98(16): 8931-2 (2001).
LeVine H III., Thioflavine T interaction with amyloid 1-sheet structures. Amyloid: Int. J. Exp. Clin. Invest., 2: 1-6, (1995).
Li et al., Improvement of a low pH antigen-antibody dissociation procedure for ELISA measurement of circulating anti-Abeta antibodies, BMC neuroscience 8: 22 (2007).
Linke et al., Morphologic, chemical and immunologic studies of amyloid-like fibrils formed from Bence Jones proteins by proteolysis, J. Immunol., 111: 10-23 (1973).
Makin et al., Molecular basis for amyloid fibril formation and stability. Proc. Natl. Acad. Sci. USA, 102: 315-20 (2005).
Makin et al., Structures for amyloid fibrils, The FEBS Journal, 272(23): 5950-61 (2005).
Malencik et al., Dityrosine as a product of oxidative stress and fluorescent probe, Amino acids, 25(3-4): 233-47 (2003).
Marks et al., By-passing immunization. Human antibodies from V-gene libraries displayed on phage. J Mol. Biol., 222(3): 581-97 (1991).
Meehan et al., Amyloid fibril formation by lens crystallin proteins and its implications for cataract formation, J. Biol. Chem., 279: 3413-9 (2004).
Merlini et al., Molecular Mechanisms of Amyloidosis, N. Engl. J. Med., 349(6): 583-96 (2003).
Moir et al., Autoantibodies to Redox-modified Oligomeric A{beta} Are Attenuated in the Plasma of Alzheimer's Disease Patients, J. Biol. Chem., 280(17): 17458-63 (2005).
Monaco et al., Cerebral amyloidoses: molecular pathways and therapeutic challenges, Curr. Med. Chem., 13(16): 1903-13 (2006).
Morgan, et al., A beta peptide vaccination prevents memory loss in an animal model of Alzheimer's disease, Nature 408(6815): 982-5 (2000).
Morrison et al., Chimeric human antibody molecules: mouse antigen-binding domains with human constant region domains, Proc. Natl. Acad. Sci. USA, 81: 6851-5 (1984).
Hartmann et al., Distinct sites of intracellular production for Alzheimer's disease A beta40/42 amyloid peptides, Nature Medicine 3(9):1016-1020 (1997).
Neuberger et al., Recombinant antibodies possessing novel effector functions, Nature, 312: 604-8 (1984).
O'Nuallain et al., Conformational Abs recognizing a generic amyloid fibril epitope, Proc. Natl. Acad. Sci. USA, 99: 1485-90 (2002).
O'Nuallain et al., The amyloid-reactive monoclonal antibody 11-IF4 binds a cryptic epitope on fibrils and partially denatured immunoglobulin light chains and inhibits fibrillogenesis. In: Grateau G, Kyle R A, Skinner M (eds.) Amyloid and Amyloidosis. Proceedings of the Xth International Congress on Amyloidosis. CRC Press. pp. 482-484 (2004).
O'Nuallain et al., Seeding specificity in amyloid growth induced by heterologous fibrils, J. Biol. Chem., 279: 17490-17499 (2004).
O'Nuallain et al., Diagnostic and therapeutic potential of amyloid-reactive IgG antibodies contained in human sera, J. Immunol., 176(11): 7071-8 (2006).
O'Nuallain et al., Kinetics and thermodynamics of amyloid assembly using a high-performance liquid chromatography-based sedimentation assay, Methods Enzymol., 413: 34-74 (2006).
O'Nuallain et al., Localization of a conformational epitope common to non-native and fibrillar immunoglobulin light chains, Biochemistry 46(5), 1240-7 (2007).
Paramithiotis et al., A prion protein epitope selective for the pathologically misfolded conformation, Nat. Med., 9: 893-9 (2003).
Riechmann et al., Expression of an antibody Fv fragment in myeloma cells, J. Mol. Biol., 203(3): 825-8 (1988).
Ross et al., Protein aggregation and neurodegenerative disease, Nat. Med., 10: S10-S17 (2004).
Schenk et al., Immunization with amyloid-beta attenuates Alzheimer-disease-like athology in the PDAPP mouse, Nature, 400(6740): 173-7 (1999).
Serpell L. C. Alzheimer's fibrils: structure and assembly, Bichim. Biophys. Acta, 1502: 16-30 (2000).
Senechal et al., Amyloid precursor protein knockdown by siRNA impairs spontaneous alternation in adult mice, Journal of neurochemistry, 102(6), 1928-40 (2007).
Seshadri et al., Fourier transform infrared spectroscopy in analysis of protein deposits, Methods Enzymol., 309: 559-76 (1999).
Solomon et al., Immunotherapy in systemic primary (AL) amyloidosis using amyloid-reactive monoclonal antibodies, Cancer Biother. Radiopharm., 18: 853-60 (2003).
Solomon, B., Clinical immunologic approaches for the treatment of Alzheimer's disease, Expert opinion on investigational drugs, 16(6): 819-28 (2007).
Souza et al., Dityrosine cross-linking promotes formation of stable alpha-synuclein polymers. Implication of nitrative and oxidative stress in the pathogenesis of neurodegenerative synucleinopathies, J. Biol. Chem., 295: 18344-49 (2000).
Stefani, M., Protein misfolding and aggregation: new examples in medicine and biology of the dark side of the protein world, Biochim. Biophys. Acta, 1739(1): 5-25 (2004).
Takeda et al., Construction of chimaeric processed immunoglobulin genes containing mouse variable and human constant region sequences, Nature, 314:452-4 (1985).
Walsh et al., Amyloid beta-protein fibrillogenesis. Detection of a protofibrillar intermediate, J. Biol. Chem., 272(35): 22364-72 (1997).
Walsh et al., Amyloid beta-protein fibrillogenesis. Structure and biological activity of protofibrillar intermediates, J. Biol. Chem., 274(36): 25945-52 (1999).
Wang et al., Clearance of amyloid-beta in Alzheimer's disease: progress, problems and perspectives, Drug Discov Today, 11(19-20): 931-8 (2006).
Watson et al., Physicochemical characteristics of soluble oligomeric Abeta and their pathologic role in Alzheimer's disease, Neurological research, 27(8): 869-81 (2005).
Weksler et al., The immune system, amyloid-beta peptide, and Alzheimer's disease, Immunological reviews, 205: 244-56 (2005).
Westermark et al., Amyloid fibril nomenclature-2002. Amyloid J. Protein Folding Disord., 9: 197-200 (2002).
Westermark et al., Amyloid: toward terminology clarification. Report from the Nomenclature Committee of the International Society of Amyloidosis, Amyloid 12(1): 1-4 (2005).
Woodhouse et al., Vaccination strategies for Alzheimer's disease: A new hope, Drugs & Aging 24(2): 107-19 (2007).
Yin Y I, Bassit B, Zhu L, Yang X, Wang C, Li Y M. (2007). Gamma-secretase substrate concentration modulates the abeta 42/abeta 40 ratio: Implications for Alzheimer's disease. J Biol Chem. Epub.
Yoburn et al., Dityrosine cross-linked Abeta peptides: fibrillar beta-structure in Abeta(1-40) is conducive to formation of dityrosine cross-links but a dityrosine cross-link in Abeta(8-14) does not induce beta-structure, Chem. Res. Toxicol., 16(4): 531-5 (2003).

1. A method of preparing crosslinked oligomers comprising incubating a peptide of an amyloid protein with horseradish peroxidase (HRP) to form a solution of cross-linked oligomers; adding copper ions to the solution to precipitate the cross-linked oligomers; and isolating the cross-linked oligomers.

2. The method of claim 1, further comprising solubilizing the peptide prior to incubation with HRP.

3. The method of claim 2, wherein the peptide is solubilized by sequential exposure to trifluoroacetic acid (TFA) and 1,1,1,3,3,3,-hexafluoro-2-propanol (HFIP) or by dissolving the peptide in sodium hydroxide (NaOH).

4. The method of claim 1, wherein the HRP is conjugated to a matrix.

5. The method of claim 4, wherein the HRP conjugated matrix is treated with a blocking agent prior to incubating with the peptide.

6. The method of claim 5, wherein the blocking agent is Bovine Serum Albumin (BSA) or gelatin.

7. The method of claim 1 further comprising incubating the precipitated cross-linked oligomers under conditions allowing removal of residual HRP and copper ions, prior to isolating the cross-linked oligomers.

8. The method of claim 7 comprising adding guanidine hydrochloride and EDTA to the precipitated cross-linked oligomers.

9. The method of claim 1, further comprising resolubilizing the precipitated cross-linked oligomers and centrifuging the resolubilized cross-linked oligomers prior to isolating the cross-linked oligomers.

10. The method of claim 1, wherein the peptide is an Aβ peptide.

11. A method of preparing soluble cross-linked oligomers comprising solubilizing the peptide of an amyloid protein by sequential exposure to trifluoroacetic acid (TFA) and 1,1,1,3,3,3,hexafluoro-2-propanol (HFIP) or by dissolving the peptide in sodium hydroxide (NaOH); and incubating the peptide with HRP to form a solution of cross-linked oligomers.

12. The method of 11, wherein the peptide is an Aβ peptide.

13. An affinity purification matrix comprising cross-linked oligomers.

14. The affinity purification matrix of claim 13, wherein the matrix comprises Sepharose.

15. The affinity purification matrix of claim 13, wherein the cross-linked oligomers are cross-linked Aβ oligomers.

16. A method of preparing an affinity purification matrix comprising purifying the cross-linked oligomers according the method of claim 1; preparing an affinity purification matrix; and conjugating the cross-linked oligomers to the matrix.

17. The method of claim 16, wherein the cross-linked oligomers are cross-linked Aβ oligomers.

18. A method of enriching a sample of oligomer reactive antibodies comprising providing an affinity purification matrix of claim 12; loading the matrix with a sample comprising oligomer reactive antibodies; and isolating the oligomer reactive antibodies.

19. The method of claim 18, wherein the sample contains IGIV or blood.

20. The method of claim 18, wherein the oligomers are dityrosine cross-linked Aβ oligomers.

21. An enriched sample of oligomer reactive antibodies.

22. A vaccine comprising oligomer reactive antibodies of claim 21.

23. A composition comprising oligomer reactive antibodies of claim 21.

24. A pharmaceutical composition comprising the oligomer reactive antibodies of claim 21.

25. The method of claim 18, wherein the oligomers reactive antibodies bind Aβ oligomers.

26. A method of generating oligomer reactive antibodies comprising using oligomers purified by the method of claim 1 as an immunogen.

27. The method of claim 26, wherein the oligomer reactive antibodies are Aβ oligomer reactive antibodies.

28. A method of treating an amyloid disorder comprising administering the oligomer reactive antibodies of claim 1 to a subject in need thereof to treat the amyloid disorder.

29. The method of claim 28, where the amyloid disorder is Alzheimer's disease, AIAPP amyloidosis, ATTR amyloidosis, or AL amyloidosis.

30. A method of screening for oligomer antibody reactivity comprising incubating a biological sample with the oligomer reactive antibodies of claim 21.

31. The method of claim 30, wherein the biological sample comprises IGIV or human blood.

32. A method of diagnosing a subject with amyloid disorder comprising obtaining a sample of bodily fluid or tissue from a subject, and incubating the sample with oligomer reactive antibodies of claim 21.

33. The method of claim 31, wherein the sample comprises human blood.

34. The method of claim 31. wherein the sample comprises human plasma.

35. The method of claim 28, wherein the oligomer reactive antibodies are Aβ oligomer reactive antibodies.

36. The method of claim 30, wherein the oligomer reactive antibodies are Aβ oligomer reactive antibodies.

37. The method of claim 32, wherein the oligomer reactive antibodies are Aβ oligomer reactive antibodies


SEQUENCE LISTING (2 sequences)

SEQ ID NO: 1 - protein, 695 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 2 - protein, 42 aa, Artificial [sequence omitted]